(19) United States
(12) Patent Application Publication
(10) Pub. No.: US 2004/0141969 A1
(43) Pub. Date: Jul. 22, 2004

(54) Method for the treatment of nephritis using anti-PDGF-DD antibodies

(76) Inventors: Juergen Floege, Aachen (DE); Gadi Gazit-Bornstein, Mountain View, CA (US); Bruce A. Keyt, Hillsborough, CA (US); William J. LaRochelle, Madison, CT (US); Henri Stephen Lichenstein, Guilford, CT (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/665,383

(22) Filed: Sep. 16, 2003

Related U.S. Application Data
(60) Provisional application No. 60/411,137, filed on Sep. 16, 2002.

(51) Int. Cl. A61K 39/395

(52) U.S. Cl. 424/145.1

(57) ABSTRACT

Embodiments of the invention described herein relate to antibodies directed to platelet derived growth factor-DD (PDGF-DD) and uses of such antibodies. The antibodies of the invention find use as diagnostics and as treatments for diseases associated with the overproduction of PDGF-DD. In particular, in accordance with embodiments of the invention, the use of anti-PDGF-DD antibodies for the treatment of nephritis and related disorders, including diseases caused by mesangial proliferation is provided.

### PRIORITY

This application claims the benefit of priority under 35 U.S.C. § 119(e) to U.S. Provisional Application No. 60/411,137, filed Sep. 16, 2002, which is hereby expressly incorporated by reference.

### FIELD OF THE INVENTION

Embodiments of the invention described herein relate to antibodies directed to platelet derived growth factor-DD (PDGF-DD) and uses of such antibodies. The antibodies of the invention find use as diagnostics and as treatments for diseases associated with the overproduction of PDGF-DD. In particular, in accordance with embodiments of the invention, the use of anti-PDGF-DD antibodies for the treatment of nephritis and related disorders, including diseases caused by mesangial proliferation is provided.

### BACKGROUND OF THE INVENTION

Nephritis is a group of kidney diseases that is a problem of growing concern in the United States and throughout the world. Nephritis can gradually progress to kidney failure that is ultimately fatal unless dialysis treatment or kidney transplantation is received. The different types of nephritis have different patterns of inheritance, and different rates of progression. Hereditary nephritis is manifested by microscopic traces of blood cells and proteins in urine, and is present and generally mild at birth. Another type of nephritis, glomerulonephritis, is an inflammation of the glomeruli, the filtering units of the kidneys. Other forms of nephritis may be sequelae of infectious disease such as mononucleosis and Streptococcus (post-infectious).

The symptoms of nephritis and other diseases related to proliferation of mesangial cells vary depending on the specific type of nephritis, but typically includes the presence of blood or proteins in the urine. In early stages of the disease, there may be no signs or symptoms. As the disease progresses, some or all of the following symptoms may occur: high blood pressure, excessive foaming of the urine, change in the color of the urine (to red or dark brown), puffiness of the eyes, hands, and feet, nausea and vomiting, difficulty breathing, and headaches. These symptoms may be used to identify the disease, to follow the course of treatment, and to identify what type of treatment is needed.

Injury to glomeruli can result in a variety of signs of the disease, including but not limited to proteinuria, hematuria, azotemia, oliguria, anuria, edema, and hypertension. The disease may also result in nephritic syndrome, acute nephritis, and rapidly progressive glomerulonephritis.

Many progressive renal diseases, including diabetic nephropathy, as well as the most frequent types of glomerulonephritides such as IgA-nephropathy are characterized by glomerular mesangial cell proliferation and/or matrix accumulation. Striker et al., Lab Invest 64:446-456 (1991). Some evidence now suggests that platelet derived growth factors (PDGFs) and the associated PDGF-system, may be involved in mesangial cell proliferation and matrix accumulation. Floege et al., supra (2001) and Floege et al., Am. J. Pathol. 154:169-79 (1999); Gilbert et al., Kidney Int. 59:1324-32 (2001); Nakamura et al., Kidney Int., 59:2134-45 (2001). In addition, both PDGF β-receptor subunit as well as PDGF B-chain are overexpressed in renal interstitial fibrosis. Kliem et al., Kidney Int. 49:666-78 (1996). Infusion of large doses of the dimer, PDGF-BB alone is able to induce interstitial fibrotic changes in normal rat kidney. Tang et al., Am. J. Pathol. 148:1169-80 (1996).

For two decades the platelet derived growth factor system consisted of only two PDGF chains, PDGF-A and -B, that are secreted as homo- or heterodimers and bind to dimeric PDGF receptors composed of α- and/or β-chains. Whereas PDGF-A binds to the α-chain only, PDGF-B is a ligand for all receptor types. Floege et al., “Growth factors and cytokines,” in Immunologic Renal Diseases (Neilson E. G. and Couser W. G., eds., 2d ed. 2001). Recently two other PDGF isoforms, designated PDGF-C and -D, were described that are released as homodimers only. According to current terminology, the homodimer form of PDGF-C is known as “PDGF-CC” and the homodimer form of PDGF-D is known as “PDGF-DD.” LaRochelle et al., Nat. Cell Biol. 3:517-21 (2001); Li et al., Nat. Cell Biol. 2:302-09 (2000); and Bergsten et al., Nat. Cell Biol. 3:512-16 (2001). The core chain of PDGF-CC appears to be largely a ligand for the αα-PDGF receptor, while PDGF-DD largely binds to the ββ-PDGF receptor. Id. In both cases, some binding has also been described to the αβ3-receptor. LaRochelle et al., supra (2001); Bergsten et al., supra (2001); Gilbertson et al., J. Biol. Chem. 276:27406-14 (2001). All four PDGF isoforms, as well as both receptor chains are expressed in the kidney, albeit in distinct spatial arrangements. Floege et al., supra (2001); Changsirikulchai et al., Kidney Int. 62(6):2043-54 (2002); Eitner et al., J. Am. Soc. Nephrol. 13(4):910-17 (2002).

PDGF-D is secreted as the disulphide-linked homodimer PDGF-DD, which is activated upon limited proteolysis with dissociation of its CUB-domain to become a specific agonistic ligand for PDGF-ββ- and αβ-receptor. In developing and in adult normal kidneys, PDGF-DD is expressed in visceral glomerular epithelial cells and some vascular smooth muscle cells. Changsirikulchai et al., supra (2002). In the developing mouse kidney, only cells of the branching ureter exhibited PDGF-DD immunoreactivity. Bergsten et al., supra (2001).

Diagnosis of nephritis is typically by identification of a family history and/or examination of the urinary sediment for the presence of red blood cells and protein, specifically for hematuria or albuminuria. Unfortunately, no specific treatment is known to affect the underlying pathological process or to alter the clinical course. Antibiotics, anticoagulants, steroids, and immunosuppressive agents have wrought no benefit. Control of hypertension is suggested and protein restriction may be of some use. When terminal uremia occurs, dialysis and even transplantation of the kidney are necessary. Thus, a novel approach for the treatment of nephritis is needed.

### SUMMARY OF THE INVENTION

Embodiments of the invention relate to the discovery that administration of anti-PDGF-DD antibodies, were highly effective at reducing proliferation of glomerular cells and of treating disorders associated with their proliferation.

Accordingly, one embodiment of the invention is the use of fully human anti-PDGF-DD antibodies, and anti-PDGF-DD antibody preparations with desirable properties from a therapeutic perspective, to inhibit the progression of nephritis and related diseases. Preferably, the antibodies have a heavy chain amino acid having a sequence selected from the group consisting of SEQ ID NOS: 2, 6, 10, 14, 18, 22, 26, 30, 34, 38, 42, 46, 50, 54, 58, 62, 66, 70, and 74. More preferably, the antibodies further have a light chain amino acid having a sequence selected from the group consisting of SEQ ID NOS: 4, 8, 12, 16, 20, 24, 28, 32, 36, 40, 44, 48, 52, 56, 60, 64, 68, and 72.

It will be appreciated that embodiments of the invention are not limited to any particular anti-PDGF-DD antibody, or any specific form of an antibody. For example, the anti-PDGF-DD antibody may be a full length antibody (e.g. having an intact human Fc region) or an antibody fragment (e.g. a Fab, Fab′ or F(ab′)₂). In addition, the antibody may be manufactured from a hybridoma that secretes the antibody, or from a recombinantly produced cell that has been transformed or transfected with a gene or genes encoding the antibody.

In a preferred embodiment, the invention includes the treatment of nephritis and related diseases in humans, including but not limited to, mesangial proliferative nephritis, mesangial proliferative glomerulonephritis, mesangiocapillary glomerulonephritis, systemic lupus erythematosus, glomerular nephritis, renal failure, and diabetic nephropathy.

In one embodiment, the anti-PDGF-DD antibody forms a pharmaceutical composition comprising an effective amount of the antibody, or a fragment thereof, in association with a pharmaceutically acceptable carrier or diluent. In an alternative embodiment, an anti-PDGF-DD antibody is linked to a radioisotope or a toxin. In another embodiment, the anti-PDGF-DD antibody or fragment thereof is conjugated to a therapeutic agent. The therapeutic agent can be a toxin or a radioisotope. Preferably, such antibodies can be used for the treatment of diseases, such as, for example, nephritis, progressive renal diseases, and related diseases, such as mesangial proliferative nephritis, mesangial proliferative glomerulonephritis, mesangiocapillary glomerulonephritis, systemic lupus erythematosus, glomerular nephritis, renal interstitial fibrosis, renal failure, and diabetic nephropathy.

In another embodiment, the invention includes a method for treating diseases or conditions associated with the expression of PDGF-DD in a patient by administering to the patient an effective amount of an anti-PDGF-DD antibody. The patient is a mammalian patient, preferably a human patient. The disease or condition can be, for example, nephritis, progressive renal diseases, and related diseases, such as mesangial proliferative nephritis, mesangial proliferative glomerulonephritis, mesangiocapillary glomerulonephritis, systemic lupus erythematosus, glomerular nephritis, renal interstitial fibrosis, renal failure, or diabetic nephropathy. Additional embodiments include methods for the treatment of diseases or conditions associated with the expression of PDGF-DD in a mammal by identifying a mammal in need of treatment for nephritis and administering to the mammal a therapeutically effective dose of anti-PDGF-DD antibodies.

Alternatively, anti-PDGF-DD antibodies may be administered to prevent a mammal from contracting diseases or conditions associated with the expression of PDGF-DD including, but not limited to, nephritis or related diseases, and diseases caused by mesangial proliferation. Preferably the anti-PDGF-DD antibodies are fully human. The disease or condition can be nephritis and related diseases, including but not limited to, nephritis, progressive renal diseases, and related diseases, such as mesangial proliferative nephritis, mesangial proliferative glomerulonephritis, mesangiocapillary glomerulonephritis, systemic lupus erythematosus, glomerular nephritis, renal interstital fibrosis, renal failure, and diabetic nephropathy.

In yet another embodiment, the invention includes a method for inhibiting cell proliferation associated with, or caused by, the expression of PDGF-DD by contacting cells expressing PDGF-DD with an effective amount of an anti-PDGF-DD antibody or a fragment thereof and incubating the cells and antibody, wherein the incubation results in inhibited proliferation of cells. In one embodiment, the cell proliferation is mesangial cell proliferation. Further, the mesangial cells can be human mesangial cells. In addition, the method can be performed in vivo.

In another embodiment, the invention is an article of manufacture including a container having a composition containing an anti-PDGF-DD antibody, and a package insert or label indicating that the composition can be used to treat conditions characterized by the overexpression of PDGF-D. Preferably a mammal and, more preferably, a human, receives the anti-PDGF-DD antibody. In a preferred embodiment, nephritis and related diseases in humans are treated, including but not limited to, nephritis, progressive renal diseases, and related diseases, such as mesangial proliferative nephritis, mesangial proliferative glomerulonephritis, mesangiocapillary glomerulonephritis, systemic lupus erythematosus, glomerular nephritis, renal interstital fibrosis, renal failure, and diabetic nephropathy.

Another embodiment is a method for identifying risk factors, of disease, diagnosis of disease, and staging of disease which involves identifying overproliferation of mesangial cells in the glomerulus using anti-PDGF-DD antibodies.

In one embodiment, the invention includes a method for diagnosing a condition associated with the expression of PDGF-DD in a cell by contacting the cell with an anti-PDGF-DD antibody, and detecting the presence of PDGF-DD. Preferred conditions include, without limitation, mesangial proliferative nephritis, mesangial proliferative glomerulonephritis, mesangiocapillary glomerulonephritis, systemic lupus erythematosus, glomerular nephritis, renal failure, and diabetic nephropathy.

In still another embodiment, the invention includes an assay kit for the detection of PDGF-DD in mammalian tissues or cells to screen for nephritis and related diseases in humans, including but not limited to, mesangial proliferative nephritis, mesangial proliferative glomerulonephritis, mesangiocapillary glomerulonephritis, systemic lupus erythematosus, glomerular nephritis, renal failure, and diabetic nephropathy. The kit includes an antibody that binds to PDGF-DD and a means for indicating the reaction of the antibody with PDGF-DD, if present. Preferably the antibody is a monoclonal antibody. In one embodiment, the antibody that binds PDGF-DD is labeled. In another embodiment the antibody is an unlabeled first antibody and the means for indicating the reaction is a labeled anti-immunoglobulin antibody. Preferably, the antibody is labeled with a marker selected from the group consisting of: a fluorochrome, an enzyme, a radionuclide and a radiopaque material.

Yet another embodiment is the use of an anti-PDGF-DD antibody in the preparation of a medicament for the treatment of nephritis and related diseases. In one embodiment, the disease is selected from the group comprising nephritis, progressive renal diseases, and related diseases, such as mesangial proliferative nephritis, mesangial proliferative glomerulonephritis, mesangiocapillary glomerulonephritis, systemic lupus erythematosus, glomerular nephritis, renal interstital fibrosis, renal failure, and diabetic nephropathy.

### BRIEF DESCRIPTION OF THE DRAWINGS

FIG. 1 shows the characterization of anti-PDGF-DD mAb 6.4 specificity by ELISA.

FIG. 2 shows the shows further characterization of anti-PDGF-DD mAb 6.4 specificity by ELISA.

FIG. 3 shows the characterization of anti-PDGF-DD mAb specificity by Western Blot Analysis.

FIG. 4 is a line graph that shows that anti-PDGF-DD mAb 6.4 was able to neutralize PDGF-DD induced BrdU incorporation in NIH3T3 cells with an IC₅₀ of approximately 75 ng/ml.

FIG. 5 is a bar chart that shows that PDGF-DD acts as a growth factor for mesangial cells in vitro. Data are means ±SD of four independent experiments. * indicates p<0.05 versus unstimulated control.

FIG. 6 is a bar chart that shows the results of PDGF-DD-induced BrdU incorporation in human mesangial cells.

FIG. 7 is a graph that shows PDGF-DD expression in human serum for patients with various types of nephritis. A closed circle represents the PDGF-DD concentration for an individual clinical serum sample. PDGF-DD serum concentrations are grouped according to the patient disease indication. The number of patients (n) for a given clinical indication is provided, along with the mean PDGF-DD concentration in ng/ml.

FIG. 8 shows immunochistochemical analysis of normal rat mesangium cells and the mesangium cells of rats with anti-Thy-1 induced nephritis. Elevated anti-PDGF-DD staining was found in rats with anti-Thy-1 induced nephritis. Mesangium, tubules and surrounding vasculature is shown. Mesangium cells included pericytes and renal tubules. White and gray arrows depict capillary and tubule staining respectively.

FIG. 9 is a line graph that shows simulated fully human mAb kinetics performed on rats. As shown, there is only a small peak to trough fluctuation expected over 4 days, even after a single dose.

FIG. 10 is a line graph that shows transcript expression of PDGF-A, -B, -C and -D in the course of anti-Thy1.1 nephritis relative to the expression in untreated rats.

FIG. 11 shows PDGF-DD protein was overexpressed during anti-Thy 1.1 nephritis in glomeruli. No PDGF-DD expression was noted in normal glomeruli (FIG. 11(A)), whereas expression can be readily detected during mesangioproliferative nephritis at day 7 after disease induction (FIG. 11(B)). No glomerular staining is present, when the anti-PDGF-DD antibody is replaced by an equal concentration of control IgG (FIG. 11(C)). Magnification is 600×.

FIGS. 12A-H are bar charts that show glomerular changes on day 5 and day 8 after disease induction in rats with mesangioproliferative anti Thy 1.1 nephritis treated with either anti-PDGF-DD antibody, irrelevant control IgG or PBS alone.

FIG. 13 is a bar graph that shows the results of glomerular proliferation as measured by BrdU incorporation in rats. Nephritic rats were treated with anti-PDGF-DD mAb 6.4, or control antibodies, or PBS. Healthy rats were treated with anti-PDGF-DD mAb 6.4 or control antibodies.

FIG. 14 is a bar graph that shows the results of glomerular proliferation as measured by PAS stain and quantitation of mitosis in rats. Nephritic rats were treated with anti-PDGF-DD mAb 6.4, or control antibodies, or PBS. Healthy rats were treated with anti-PDGF-DD mAb 6.4 or control antibodies.

FIG. 15 is a bar graph that demonstrates the effect of anti-PDGF-DD mAb 6.4 on mesangial cell mitosis in an acute rat anti-Thy-1 model. Anti-Thy-1 rats were treated with anti-PDGF-DD mAb 6.4, or control antibodies, or PBS. Healthy rats were treated with anti-PDGF-DD mAb 6.4 or control antibodies.

FIG. 16 is a bar graph that demonstrates the dose-responsive effects of anti-PDGF-DD mAb 6.4 on mitosis in glomerular cells in an acute rat Thy-1 model.

FIG. 17 is a bar graph that demonstrates the dose-responsive effects of anti-PDGF-DD mAb 6.4 on BrdU incorporation in an acute rat Thy-1 model.

FIG. 18 shows the immunohistochemical analysis of normal and diseased human kidney tissue. Mesangium, tubules and surrounding vasculature is shown. White and gray arrows depict capillary and tubule staining respectively. Small black arrows show punctate inflammatory cell deposits in mesangium.

### DETAILED DESCRIPTION

The invention described herein relates to methods for effectively treating, diagnosing, and/or staging nephritis and related conditions. Such conditions include mesangial proliferative nephritis, mesangial proliferative glomerulonephritis, mesangiocapillary glomerulonephritis, systemic lupus erythematosus, glomerular nephritis, renal failure, and diabetic nephropathy. In one particular embodiment, the invention includes administering a therapeutically effective amount of anti-PDGF-DD antibodies as a treatment for nephritis and related conditions. In preferred embodiments, the antibodies are fully human antibodies against the dimer PDGF-DD.

Other embodiments of the invention relate to other compounds that result in a reduction of mesangioproliferative changes in vivo. Thus, compounds that reduce the level of PDGF-DD would be useful in treatment of nephritis. PDGF-D nucleic acids, polypeptides, antibodies, agonists, antagonists, and other related compound's uses are disclosed more fully below.

As described above, PDGF-D signals through a PDGF-B receptor and is mitogenic for rat mesangial cells (MC). Low levels of PDGF-D mRNA were detected in normal rat glomeruli. However, incubation of cultured rat MCs with 100 ng/ml PDGF-DD led to a 7-fold increase in MC proliferation with a maximum after 24 hours. By real-time PCR, PDGF-D mRNA was detected in both cultured mesangial cells and glomeruli isolated from normal rat kidney. Following the induction of mesangioproliferative anti-Thy 1.1 nephritis in rats, glomerular PDGF-D mRNA and protein expression increased significantly from days 4 to 9 in comparison to non-nephritic rats as determined by real time PCR. Peak expression of PDGF-D mRNA occurred 2 days later than peak PDGF-B mRNA expression. Additionally, PDGF-DD serum levels increased significantly in the nephritic animals on day 7.

To investigate the functional role of PDGF-DD during the nephritis, neutralizing fully human monoclonal anti-PDGF-DD antibodies were generated in Xenomouse® (Abgenix, Inc., Fremont, Calif.). Following the induction of anti-Thy 1.1 nephritis, rats were treated on day 3 and day 5 after disease induction with 10 and 4 mg/kg fully human anti-PDGF-DD antibody mAb 6.4 (n=15) or irrelevant human monoclonal antibody (n=15) or PBS (n=15) by daily intraperitoneal injection. On day 8 after disease induction antagonism of PDGF-DD led to a significant reduction of mitotic figures per 100 glomeruli (anti-PDGF-DD: 9.9+0.9; irrelevant IgG: 13.9+0.9; PBS: 14.7±1.0; p<0.0014) as well as of glomerular cells incorporating the thymidine analog BrdU (anti-PDGF-DD mAb 6.4: 1.62±0.23; irrelevant IgG: 2.88±0.28; PBS: 2.91+0.18; p<0.0016). Reduction of glomerular cell proliferation in the rats receiving anti-PDGF-DD was not associated with reduced glomerular expression of PDGF-B mRNA as determined by real time PCR.

Injection of anti-PDGF-DD antibodies into normal rats did not affect the physiologic glomerular cell turnover as compared to normal rats receiving irrelevant IgG. Thus, PDGF-DD, produced by glomerular mesangial cells acts as a glomerular cell mitogen both in vitro and in vivo.

Sequence Listing

The heavy chain and light chain variable region nucleotide and amino acid sequences of representative human anti-PDGF-DD antibodies are provided in the sequence listing, the contents of which are summarized in Table 1 below.

TABLE 1

| mAb | | SEQ ID |
|---|---|---|
| ID No.: | Sequence | NO: |
| 6.4 | Nucleotide sequence encoding the variable region of the heavy chain | 1 |
| | Amino acid sequence encoding the variable region of the heavy chain | 2 |
| | Nucleotide sequence encoding the variable region of the light chain | 3 |
| | Amino acid sequence encoding the variable region of the light chain | 4 |
| 1.6 | Nucleotide sequence encoding the variable region of the heavy chain | 5 |
| | Amino acid sequence encoding the variable region of the heavy chain | 6 |
| | Nucleotide sequence encoding the variable region of the light chain | 7 |
| | Amino acid sequence encoding the variable region of the light chain | 8 |
| 1.11 | Nucleotide sequence encoding the variable region of the heavy chain | 9 |
| | Amino acid sequence encoding the variable region of the heavy chain | 10 |
| | Nucleotide sequence encoding the variable region of the light chain | 11 |
| | Amino acid sequence encoding the variable region of the light chain | 12 |
| 1.17 | Nucleotide sequence encoding the variable region of the heavy chain | 13 |
| | Amino acid sequence encoding the variable region of the heavy chain | 14 |
| | Nucleotide sequence encoding the variable region of the light chain | 15 |
| | Amino acid sequence encoding the variable region of the light chain | 16 |
| 1.18 | Nucleotide sequence encoding the variable region of the heavy chain | 17 |
| | Amino acid sequence encoding the variable region of the heavy chain | 18 |
| | Nucleotide sequence encoding the variable region of the light chain | 19 |
| | Amino acid sequence encoding the variable region of the light chain | 20 |
| 1.19 | Nucleotide sequence encoding the variable region of the heavy chain | 21 |
| | Amino acid sequence encoding the variable region of the heavy chain | 22 |
| | Nucleotide sequence encoding the variable region of the light chain | 23 |
| | Amino acid sequence encoding the variable region of the light chain | 24 |
| 1.23 | Nucleotide sequence encoding the variable region of the heavy chain | 25 |
| | Amino acid sequence encoding the variable region of the heavy chain | 26 |
| | Nucleotide sequence encoding the variable region of the light chain | 27 |
| | Amino acid sequence encoding the variable region of the light chain | 28 |
| 1.24.1 | Nucleotide sequence encoding the variable region of the heavy chain | 29 |
| | Amino acid sequence encoding the variable region of the heavy chain | 30 |
| | Nucleotide sequence encoding the variable region of the light chain | 31 |
| | Amino acid sequence encoding the variable region of the light chain | 32 |
| 1.25.1 | Nucleotide sequence encoding the variable region of the heavy chain | 33 |
| | Amino acid sequence encoding the variable region of the heavy chain | 34 |
| | Nucleotide sequence encoding the variable region of the light chain | 35 |
| | Amino acid sequence encoding the variable region of the light chain | 36 |
| 1.29 | Nucleotide sequence encoding the variable region of the heavy chain | 37 |
| | Amino acid sequence encoding the variable region of the heavy chain | 38 |
| | Nucleotide sequence encoding the variable region of the light chain | 39 |
| | Amino acid sequence encoding the variable region of the light chain | 40 |
| 1.33 | Nucleotide sequence encoding the variable region of the heavy chain | 41 |
| | Amino acid sequence encoding the variable region of the heavy chain | 42 |
| | Nucleotide sequence encoding the variable region of the light chain | 43 |
| | Amino acid sequence encoding the variable region of the light chain | 44 |
| 1.38.1 | Nucleotide sequence encoding the variable region of the heavy chain | 45 |
| | Amino acid sequence encoding the variable region of the heavy chain | 46 |
| | Nucleotide sequence encoding the variable region of the light chain | 47 |
| | Amino acid sequence encoding the variable region of the light chain | 48 |
| 1.39.1 | Nucleotide sequence encoding the variable region of the heavy chain | 49 |
| | Amino acid sequence encoding the variable region of the heavy chain | 50 |
| | Nucleotide sequence encoding the variable region of the light chain | 51 |
| | Amino acid sequence encoding the variable region of the light chain | 52 |
| 1.45 | Nucleotide sequence encoding the variable region of the heavy chain | 53 |
| | Amino acid sequence encoding the variable region of the heavy chain | 54 |
| | Nucleotide sequence encoding the variable region of the light chain | 55 |
| | Amino acid sequence encoding the variable region of the light chain | 56 |
| 1.46.1 | Nucleotide sequence encoding the variable region of the heavy chain | 57 |
| | Amino acid sequence encoding the variable region of the heavy chain | 58 |
| | Nucleotide sequence encoding the variable region of the light chain | 59 |
| | Amino acid sequence encoding the variable region of the light chain | 60 |
| 1.48.1 | Nucleotide sequence encoding the variable region of the heavy chain | 61 |
| | Amino acid sequence encoding the variable region of the heavy chain | 62 |
| | Nucleotide sequence encoding the variable region of the light chain | 63 |
| | Amino acid sequence encoding the variable region of the light chain | 64 |
| 1.49.1 | Nucleotide sequence encoding the variable region of the heavy chain | 65 |
| | Amino acid sequence encoding the variable region of the heavy chain | 66 |
| | Nucleotide sequence encoding the variable region of the light chain | 67 |
| | Amino acid sequence encoding the variable region of the light chain | 68 |
| 1.51 | Nucleotide sequence encoding the variable region of the heavy chain | 69 |
| | Amino acid sequence encoding the variable region of the heavy chain | 70 |
| | Nucleotide sequence encoding the variable region of the light chain | 71 |
| | Amino acid sequence encoding the variable region of the light chain | 72 |
| 1.40.1 | Nucleotide sequence encoding the variable region of the heavy chain | 73 |
| | Amino acid sequence encoding the variable region of the heavy chain | 74 |
| 1.22 | Nucleotide sequence encoding the variable region of the heavy chain | 75 |
| | Amino acid sequence encoding the variable region of the heavy chain | 76 |
| | Nucleotide sequence encoding the variable region of the light chain | 77 |
| | Amino acid sequence encoding the variable region of the light chain | 78 |
| 1.59 | Nucleotide sequence encoding the variable region of the heavy chain | 79 |
| | Amino acid sequence encoding the variable region of the heavy chain | 80 |
| | Nucleotide sequence encoding the variable region of the light chain | 81 |
| | Amino acid sequence encoding the variable region of the light chain | 82 |

Definitions

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (3d ed., Cold Spring Harbor Laboratory Press, Cold Spring Harbor, N.Y. (2001)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the embodiments provided herein, the following terms, unless otherwise indicated, shall be understood to have the following meanings:

Mesangial cells are cells found within the glomerular lobules of mammalian kidney where they serve as structural supports, may regulate blood flow, are phagocytic and may act as accessory cells, presenting antigen in immune responses.

Mesangial proliferative nephritis is glomerulonephritis with an increase in glomerular mesangial cells or matrix, or mesangial deposits.

Mesangial proliferative glomerulonephritis is an inflammation of the kidney glomerulus (blood filtering portion of the kidney) due to the abnormal deposition of IgM antibody in the mesangium layer of the glomerular capillary.

Mesangiocapillary glomerulonephritis is a kidney disorder which results in kidney dysfunction. Inflammation of the glomeruli result from an abnormal immune response and the deposition of antibodies within the kidney (glomerulus). Symptoms include cloudy urine (pyuria), decreased urine output, swelling and hypertension. The disorder often results in end-stage renal disease.

The mesangium is the central part of the glomerulus between capillaries. Mesangial cells are phagocytic and for the most part separated from capillary lumina by endothelial cells. Extraglomerular mesangium are mesangial cells that fill the triangular space between the macula densa and the afferent and efferent arterioles of the juxtaglomerular apparatus.

Glomerulonephritis is a variety of nephritis which is characterized by inflammation of the capillary loops in the glomeruli of the kidney. It occurs in acute, subacute and chronic forms and may be secondary to infection or autoimmune disease.

The term “PDGF-DD” includes PDGF-DD in its full length and mature form, along with its variants, and fragments thereof. Accordingly, PDGF-DD can include, but is not limited to, variants CG52053-01, CG52053-02, CG52053-03, CG52053-04, CG52053-05, CG52053-06, and CG52053-07. (CuraGen, New Haven, Conn.). More information can be found in PCT Publication WO 01/25433 filed Oct. 7, 1999.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g. free of murine proteins, (3) is expressed by a cell from a different species, or (4) does not occur in nature.

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise the human heavy chain immunoglobulin molecules and the human kappa light chain immunoglobulin molecules, as well as antibody molecules formed by combinations comprising the heavy chain immunoglobulin molecules with light chain immunoglobulin molecules, such as the kappa light chain immunoglobulin molecules, and vice versa, as well as fragments and analogs thereof.

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See M. O. Dayhoff, in Atlas of Protein Sequence and Structure, Vol. 5, 101-110 and Supplement 2 to Vol. 5, 1-10 (National Biomedical Research Foundation 1972). The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a “GTATA”.

The following terms are used to describe the sequence relationships between two or more polynucleotide or amino acid sequences: “reference sequence,” “comparison window,” “sequence identity,” “percentage of sequence identity,” and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window,” as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or 1) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2d ed., Golub, E. S. and Gren, D. R. eds., Sinauer Associates, Sunderland, Mass. 1991), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the invention described herein. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the invention described herein, providing that the variations in the amino acid sequence maintain at least 75%, more preferably at least 80%, 90%, 95%, and most preferably 99% of the originial sequence. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al., Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally occurring peptide sequence. For example, single or multiple amino acid substitutions (preferably conservative amino acid substitutions) may be made in the naturally occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, ed., W. H. Freeman and Company, New York 1984); Introduction to Protein Structure (Branden, C. and Tooze, J. eds., Garland Publishing, New York, N.Y. 1991); and Thornton et al., Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to a PDGF-DD dimer, under suitable binding conditions, (2) ability to block appropriate PDGF-DD binding, or (3) ability to inhibit PDGF-DD expressing cell growth in vitro or in vivo. Typically, polypeptide analogs comprise a conservative amino acid substitution (or addition or deletion) with respect to the naturally occurring sequence. Analogs typically are at least 20 amino acids long, preferably at least 50 amino acids long or longer, and can often be as long as a full-length naturally occurring polypeptide.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. Adv. Drug Res. 15:29 (1986); Veber and Freidinger, TINS p.392 (1985); and Evans et al., J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the dissociation constant is ≦1 μM, preferably ≦100 nM and most preferably ≦10 nM.

The term “agent” is used herein to denote a chemical compound, a mixture of chemical compounds, a biological macromolecule, or an extract made from biological materials.

“Active” or “activity” for the purposes herein refers to form(s) of PDGF-DD polypeptide which retain a biological and/or an immunological activity of native or naturally occurring PDGF-DD polypeptides, wherein “biological” activity refers to a biological function (either inhibitory or stimulatory) caused by a native or naturally occurring PDGF-DD polypeptide other than the ability to induce the production of an antibody against an antigenic epitope possessed by a native or naturally occurring PDGF-DD polypeptide and an “immunological” activity refers to the ability to induce the production of an antibody against an antigenic epitope possessed by a native or naturally occurring PDGF-DD polypeptide.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Mammal” refers to any animal classified as a mammal, including humans, other primates, such as monkeys, chimpanzees and gorillas, domestic and farm animals, and zoo, sports, laboratory, or pet animals, such as dogs, cats, cattle, horses, sheep, pigs, goats, rabbits, rodents, etc. For purposes of treatment, the mammal is preferably human.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an “F(ab′)₂” fragment that has two antigen-combining sites and is still capable of cross-linking antigen.

“Fv” is the minimum antibody fragment that contains a complete antigen-recognition and binding site of the antibody. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, for example, even a single variable domain (e.g., the VH or VL portion of the Fv dimer or half of an Fv comprising only three CDRs specific for an antigen) may have the ability to recognize and bind antigen, although, possibly, at a lower affinity than the entire binding site.

A Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. F(ab′)₂ antibody fragments originally were produced as pairs of Fab′ fragments which have hinge cysteines between them. Other chemical couplings of antibody fragments are also known.

“Solid phase” means a non-aqueous matrix to which the antibodies described herein can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phases can comprise the well of an assay plate; in others it is a purification column (e.g., an affinity chromatography column). This term also includes a discontinuous solid phase of discrete particles, such as those described in U.S. Pat. No. 4,275,149.

The term “liposome” is used herein to denote a small vesicle composed of various types of lipids, phospholipids and/or surfactant which is useful for delivery of a drug (such as a PDGF-DD polypeptide or antibody thereto) to a mammal. The components of the liposomes are commonly arranged in a bilayer formation, similar to the lipid arrangement of biological membranes.

The term “small molecule” is used herein to describe a molecule with a molecular weight below about 500 Daltons.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “patient” includes human and veterinary subjects.

Anti-PDGF-DD Antibodies

Antibodies, or parts, fragments, mimetics, or derivatives thereof, may be any type of antibody or part which recognizes a PDGF-DD dimer. In certain embodiments, it is preferred that the antibody, or part thereof, can neutralize PDGF-DD. In additional embodiments it is preferred that the antibody, or part thereof, can reduce the symptoms associated with PDGF-DD and nephritis, including but not limited to inflammation, fluid retention, tissue swelling, pain, puffiness, high blood pressure, brain swelling, visual disturbances, low urine volume, and urine containing blood. According to one embodiment, the antibody can be anti-PDGF-DD mAb 6.4, for example. Further examples of such antibodies can be found in related U.S. patent application Ser. No. 10/041,860, filed Jan. 7, 2002.

Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2d ed. Raven Press, N.Y. (1989)) (incorporated by reference in its entirety for all purposes). The variable regions of each light/heavy chain pair form the antibody binding site. Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79: 315-321 (1990), Kostelny et al., J. Immunol. 148:1547-1553 (1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

It will be appreciated that such bifunctional or bispecific antibodies are contemplated and encompassed by the invention.

Human Antibodies and Humanization of Antibodies

Embodiments of the invention described herein also contemplate and encompass human antibodies. For treatment of a human, human antibodies avoid certain of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, it has been postulated that one can develop humanized antibodies or generate fully human antibodies through the introduction of human antibody function into a rodent so that the rodent would produce fully human antibodies.

Human Antibodies

One method for generating fully human antibodies is through the use of XenoMouse® strains of mice that have been engineered to contain human heavy chain and light chain genes within their genome. For example, a XenoMouse® mouse containing 245 kb and 190 kb-sized germline configuration fragments of the human heavy chain locus and kappa light chain locus is described in Green et al., Nature Genetics 7:13-21 (1994). The work of Green et al. was extended to the introduction of greater than approximately 80% of the human antibody repertoire through utilization of megabase-sized, germline configuration YAC fragments of the human heavy chain loci and kappa light chain loci, respectively. See Mendez et al., Nature Genetics 15:146-56 (1997) and U.S. patent application Ser. No. 08/759,620, filed Dec. 3, 1996, the disclosures of which are hereby incorporated by reference. Further, XenoMouse® mice have been generated that contain the entire lambda light chain locus (U.S. Patent Application Serial No. 60/334,508, filed Nov. 30, 2001). And, XenoMouse® mice have been generated that produce multiple isotypes (see, e.g., WO 00/76310). XenoMouse® strains are available from Abgenix, Inc. (Fremont, Calif.).

The production of XenoMouse® mice is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med., 188:483-495 (1998). See also European Patent No., EP 463,151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. No. 5,591,669 and 6,023,010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

The inventors of Surani et al., cited above and assigned to the Medical Research Counsel (the “MRC”), produced a transgenic mouse possessing an Ig locus through use of the minilocus approach. The inventors on the GenPharm International work, cited above, Lonberg and Kay, following the lead of the present inventors, proposed inactivation of the endogenous mouse Ig locus coupled with substantial duplication of the Surani et al. work.

An advantage of the minilocus approach is the rapidity with which constructs including portions of the Ig locus can be generated and introduced into animals. Commensurately, however, a significant disadvantage of the minilocus approach is that, in theory, insufficient diversity is introduced through the inclusion of small numbers of V, D, and J genes. Indeed, the published work appears to support this concern. B-cell development and antibody production of animals produced through use of the minilocus approach appear stunted. Therefore, research surrounding the invention described herein has consistently been directed towards the introduction of large portions of the Ig locus in order to achieve greater diversity and in an effort to reconstitute the immune repertoire of the animals.

Kirin has also demonstrated the generation of human antibodies from mice in which, through microcell fusion, large pieces of chromosomes, or entire chromosomes, have been introduced. See European Patent Application Nos.: 773 288 and 843 961, the disclosures of which are hereby incorporated by reference.

Lidak Pharmaceuticals (now Xenorex) has also demonstrated the generation of human antibodies in SCID mice modified by injection of non-malignant mature peripheral leukocytes from a human donor. The modified mice exhibit an immune response characteristic of the human donor upon stimulation with an immunogen, which consists of the production of human antibodies. See U.S. Pat. Nos. 5,476,996 and 5,698,767, the disclosures of which are herein incorporated by reference.

Human anti-mouse antibody (HAMA) responses have led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against PDGF-DD in order to vitiate concerns and/or effects of HAMA or HACA response.

Humanization and Display Technologies

As discussed above in connection with human antibody generation, there are advantages to producing antibodies with reduced immunogenicity. To a degree, this can be accomplished in connection with techniques of humanization and display techniques using appropriate libraries. It will be appreciated that murine antibodies or antibodies from other species can be humanized or primatized using techniques well known in the art. See e.g., Winter and Harris, Immunol Today 14:43-46 (1993) and Wright et al., Crit, Reviews in Immunol. 12:125-168 (1992). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (Liu et al., P.N.A.S. 84:3439 (1987) and J. Immunol. 139:3521 (1987)). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al., “Sequences of Proteins of Immunological Interest,” N.I.H. publication no. 91-3242 (1991). Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

Antibody fragments, such as Fv, F(ab′).sub.2 and Fab may be prepared by cleavage of the intact protein, e.g., by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of heavy and light J regions may be used to design oligonucleotides for use as primers to introduce useful restriction sites into the J region for subsequent linkage of V region segements to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g., SV-40 early promoter, (Okayama et al., Mol. Cell. Bio. 3:280 (1983)), Rous sarcoma virus LTR (Gorman et al., P.N.A.S. 79:6777 (1982)), and moloney murine leukemia virus LTR (Grosschedl et al., Cell 41:885 (1985)). Also, as will be appreciated, native Ig promoters and the like may be used.

Further, human antibodies or antibodies from other species can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art. Wright and Harris, supra., Hanes and Plucthau, PNAS USA 94:4937-4942 (1997) (ribosomal display), Parmley and Smith, Gene 73:305-318 (1988) (phage display), Scott, TIBS 17:241-245 (1992), Cwirla et al., PNAS USA 87:6378-6382 (1990), Russel et al., Nucl. Acids Res. 21:1081-1085 (1993), Hoganboom et al., Immunol. Reviews 130:43-68 (1992), Chiswell and McCafferty, TIBTECH 10:80-84 (1992), and U.S. Pat. No. 5,733,743. If display technologies are utilized to produce antibodies that are not human, such antibodies can be humanized as described above.

Using these techniques, antibodies can be generated to PDGF-DD expressing cells, PDGF-DD itself, forms of PDGF-DD, epitopes or peptides thereof, and expression libraries thereto (see e.g. U.S. Pat. No. 5,703,057) which can thereafter be screened as described above for the activities described above.

Preparation of Antibodies

Through use of XenoMouse® technology, fully human monoclonal antibodies specific for the dimer form of PDGF-D were produced. Essentially, XenoMouse™ lines of mice were immunized with PDGF-DD; or fragements thereof, lymphatic cells (such as B-cells) were recovered from the mice that express antibodies, recovered cells were fused with a myeloid-type cell line to prepare immortal hybridoma cell lines, and such hybridoma cell lines were screened and selected to identify hybridoma cell lines that produced antibodies specific to PDGF-DD. Further, a characterization of the antibodies produced by such cell lines is described herein, including nucleotide and amino acid sequence analyses of the heavy and light chains of such antibodies.

In preferred embodiments the antibody is selected from neutralizing anti-PDGF-DD mAbs 1.6, 1.9, 1.18, 1.19, 1.22, 1.29, 1.33, 1.40.1, 1.45, 1.46, 1.51, 1.59, and 6.4 described herein. See PCT publication WO 03/057,857, dated Jul. 17, 2003, which is hereby experssly incorporated by reference in its entirety. Of course, the disclosed methods are not limited to use of any particular anti-PDGF-DD monoclonoal antibody, but rather encompass the use of any such antibody.

Alternatively, instead of being fused to myeloma cells to generate hybridomas, the recovered cells, isolated from immunized XenoMouse™ lines of mice, can be screened further for reactivity against the initial antigen, preferably PDGF-DD protein. Such screening includes ELISA with PDGF-DD-His protein, a competition assay with known antibodies that bind the antigen of interest, and in vitro binding to transiently transfected CHO cells expressing full length PDGF-DD. Single B cells secreting antibodies of interest are then isolated using a PDGF-DD-specific hemolytic plaque assay (Babcook et al., Proc. Natl. Acad. Sci. USA, 93:7843-7848 (1996)). Cells targeted for lysis are preferably sheep red blood cells (SRBCs) coated with the PDGF-DD antigen. In the presence of a B cell culture secreting the immunoglobulin of interest and complement, the formation of a plaque indicates specific PDGF-DD-mediated lysis of the target cells. The single antigen-specific plasma cell in the center of the plaque can be isolated and the DNA that encodes the antibody can then be isolated from the single plasma cell. Using reverse-transcriptase PCR, the DNA encoding the variable region of the antibody secreted can be specifically cloned. Such cloned DNA can then be further inserted into a suitable expression vector, preferably a vector cassette such as a pcDNA, more preferably such a pcDNA vector containing the constant domains of immunglobulin heavy and light chain. The generated vector can then be transfected into host cells, preferably CHO cells, and cultured in conventional nutrient media modified as appropriate for inducing promoters, selecting transformants, or amplifying the genes encoding the desired sequences. The isolation of multiple single plasma cells that produce antibodies specific to PDGF-DD is described herein. Further, the genetic material that encodes the specificity of the anti-PDGF-DD antibody is isolated, introduced into a suitable expression vector which is then transfected into host cells.

In general, it was found that antibodies produced by the above-mentioned cell lines possessed fully human IgG2 heavy chains with human kappa light chains. The antibodies had high affinities, typically possessing Kd's of from about 10⁻⁶ through about 10⁻¹¹ M, when measured by either solid phase and solution phase. These mAbs can be stratified into groups or “bins” based on antigen binding competition studies. See PCT publication WO 03/048,731, dated Jun. 12, 2003, which is hereby expressly incorporated by reference, for a description of this process.

Regarding the importance of affinity to therapeutic utility of anti-PDGF-DD antibodies, it will be understood that one can generate anti-PDGF-DD antibodies, for example, combinatorially, and assess such antibodies for binding affinity. One approach that can be utilized is to take the heavy chain cDNA from an antibody, prepared as described above and found to have good affinity to PDGF-DD, and combine it with the light chain cDNA from a second antibody, prepared as described above and also found to have good affinity to PDGF-DD, to produce a third antibody. The affinities of the resulting third antibodies can be measured as described herein and those with desirable dissociation constants are isolated and characterized. Alternatively, the light chain of any of the antibodies described above can be used as a tool to aid in the generation of a heavy chain that when paired with the light chain will exhibit a high affinity for PDGF-DD, or vice versa. These heavy chain variable regions in this library could be isolated from naïve animals, isolated from hyperimmune animals, generated artificially from libraries containing variable heavy chain sequences that differ in the CDR regions, or generated by any other methods that produce diversity within the CDR regions of any heavy chain variable region gene (such as random or directed mutagenesis). These CDR regions, and in particular CDR3, may be a significantly different length or sequence identity from the heavy chain initially paired with the original antibody. The resulting library could then be screened for high affinity binding to PDGF-DD to generate a therapeutically relevant antibody molecule with similar properties as the original antibody (high affinity and neutralization). A similar process using the heavy chain or the heavy chain variable region can be used to generate a therapeutically relevant antibody molecule with a unique light chain variable region. Furthermore, the novel heavy chain variable region, or light chain variable region, can then be used in a similar fashion as described above to identify a novel light chain variable region, or heavy chain variable region, that allows the generation of a novel antibody molecule.

Another combinatorial approach that can be utilized is to perform mutagenesis on germ line heavy and/or light chains that are demonstrated to be utilized in the antibodies in accordance with the invention described herein, particularly in the complementarity determining regions (CDRs). The affinities of the resulting antibodies can be measured as described herein and those with desirable dissociation constants isolated and characterized. Upon selection of a preferred binder, the sequence or sequences encoding the same may be used to generate recombinant antibodies as described above. Appropriate methods of performing mutagenesis on an oligonucleotide are known to those skilled in the art and include chemical mutagenesis, for example, with sodium bisulfite, enzymatic misincorporation, and exposure to radiation. It is understood that the invention described herein encompasses antibodies with substantial identity, as defined herein, to the antibodies explicitly set forth herein, whether produced by mutagenesis or by any other means. Further, antibodies with conservative or non-conservative amino acid substitutions, as defined herein, made in the antibodies explicitly set forth herein, are included in embodiments of the invention described herein.

Another combinatorial approach that can be used is to express the CDR regions, and in particular CDR3, of the antibodies described above in the context of framework regions derived from other variable region genes. For example, CDR1, CDR2, and CDR3 of the heavy chain of one anti-PDGF-DD antibody could be expressed in the context of the framework regions of other heavy chain variable genes. Similarly, CDR1, CDR2, and CDR3 of the light chain of an anti-PDGF-DD antibody could be expressed in the context of the framework regions of other light chain variable genes. In addition, the germline sequences of these CDR regions could be expressed in the context of other heavy or light chain variable region genes. The resulting antibodies can be assayed for specificity and affinity and may allow the generation of a novel antibody molecule.

As will be appreciated, antibodies prepared in accordance with the invention described herein can be expressed in cell lines other than hybridoma cell lines. Sequences encoding particular antibodies can be used for transformation of a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introduction of heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with constitutive PDGF-DD binding properties.

Additional Criteria for Antibody Therapeutics

As discussed herein, the function of the PDGF-DD antibody appears important to at least a portion of its mode of operation. By function, is meant, by way of example, the activity of the anti-PDGF-DD antibody in response to PDGF-DD. Accordingly, in certain respects, it may be desirable in connection with the generation of antibodies as therapeutic candidates against PDGF-DD that the antibodies may be made capable of effector function, including complement-dependent cytotoxicity (CDC) and antibody-dependent cellular cytotoxicity (ADCC). There are a number of isotypes of antibodies that are capable of the same, including, without limitation, the following: murine IgM, murine IgG2a, murine IgG2b, murine IgG3, human IgM, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see, e.g., U.S. Pat. No. 4,816,397 and U.S. Pat. No. 6,331,415), cell-cell fusion techniques (see, e.g., U.S. Pat. Nos. 5,916,771 and 6,207,418), among others.

In the cell-cell fusion technique, a myeloma or other cell line is prepared that possesses a heavy chain with any desired isotype and another myeloma or other cell line is prepared that possesses the light chain. Such cells can, thereafter, be fused and a cell line expressing an intact antibody can be isolated.

By way of example, the anti-PDGF-DD antibodies discussed herein are human anti-PDGF-DD IgG2 and IgG4 antibodies. If such antibody possessed desired binding to the PDGF-DD molecule, it could be readily isotype switched to generate a human IgM, human IgG1, or human IgG3, IgA1 or IgGA2 isotypes, while still possessing the same variable region (which defines the antibody's specificity and some of its affinity). Such molecule would then be capable of fixing complement and participating in CDC.

Accordingly, as antibody candidates are generated that meet desired “structural” attributes as discussed above, they can generally be provided with at least certain of the desired “functional” attributes through isotype switching.

Epitope Mapping

### Immunoblot Analysis

The binding of the antibodies described herein to PDGF-DD can be examined by a number of methods. For example, PDGF-DD may be subjected to SDS-PAGE and analyzed by immunoblotting. The SDS-PAGE may be performed either in the absence or presence of a reduction agent. Such chemical modifications may result in the methylation of cysteine residues. Accordingly, it is possible to determine whether the PDGF-DD antibodies described herein bind to a linear epitope on PDGF-DD.

### Surface-Enhanced Laser Desorption/Ionization

Epitope mapping of the epitope for the PDGF-DD antibodies described herein can also be performed using SELDI. SELDI ProteinChip® arrays are used to define sites of protein-protein interaction. Antigens are specifically captured on antibodies covalently immobilized onto the Protein Chip array surface by an initial incubation and wash. The bound antigens can be detected by a laser-induced desorption process and analyzed directly to determine their mass. Such fragments of the antigen that bind are designated as the “epitope” of a protein.

The SELDI process enables individual components within complex molecular compositions to be detected directly and mapped quantitatively relative to other components in a rapid, highly-sensitive and scalable manner. SELDI utilizes a diverse array of surface chemistries to capture and present large numbers of individual protein molecules for detection by a laser-induced desorption process. The success of the SELDI process is defined in part by the miniaturization and integration of multiple functions, each dependent on different technologies, on a surface (“chip”). SELDI BioChips and other types of SELDI probes are surfaces “enhanced” such that they become active participants in the capture, purification (separation), presentation, detection, and characterization of individual target molecules (e.g., proteins) or population of molecules to be evaluated.

A single SELDI protein BioChip, loaded with only the original sample, can be read thousands of times. The SELDI protein BioChips from LumiCyte hold as many as 10,000 addressable protein docking locations per 1 square centimeter. Each location may reveal the presence of dozens of individual proteins. When the protein composition information from each location is compared and unique information sets combined, the resulting composition map reveals an image with sets of features that are used collectively to define specific patterns or molecular “fingerprints.” Different fingerprints may be associated with various stages of health, the onset of disease, or the regression of disease associated with the administration of appropriate therapeutics.

The SELDI process may be described in further detail in four parts. Initially, one or more proteins of interest are captured or “docked” on the ProteinChip Array, directly from the original source material, without sample preparation and without sample labeling. In a second step, the “signal-to-noise” ratio is enhanced by reducing the chemical and biomolecular “noise.” Such “noise” is reduced through selective retention of target on the chip by washing away undesired materials. Further, one or more of the target protein(s) that are captured are read by a rapid, sensitive, laser-induced process (SELDI) that provides direct information about the target (molecular weight). Lastly, the target protein at any one or more locations within the array may be characterized in situ by performing one or more on-the-chip binding or modification reactions to characterize protein structure and function.

### Phage Display

The epitope for the PDGF-DD antibodies described herein can be determined by exposing the ProteinChip Array to a combinatorial library of random peptide 12-mer displayed on Filamentous phage (New England Biolabs).

Phage display describes a selection technique in which a peptide is expressed as a fusion with a coat protein of a bacteriophage, resulting in display of the fused protein on the surface of the virion. Panning is carried out by incubation of a library of phage displayed peptide with a plate or tube coated with the target, washing away the unbound phage, and eluting the specifically bound phage. The eluted phage is then amplified and taken through additional binding and amplification cycles to enrich the pool in favor of binding sequences. After three or four rounds, individual clones binding are further tested for binding by phage ELISA assays performed on antibody-coated wells and characterized by specific DNA sequencing of positive clones.

After multiple rounds of such panning against the PDGF-DD antibodies described herein, the bound phage may be eluted and subjected to further studies for the identification and characterization of the bound peptide.

PDGF-DD Agonists and Antagonists

Embodiments of the invention described herein also pertain to variants of a PDGF-DD protein that function as either PDGF-DD agonists (mimetics) or as PDGF-DD antagonists. Preferably, the variants of PDGF-DD protein are useful for the treatment of nephritis. Variants of a PDGF-DD protein can be generated by mutagenesis, e.g., discrete point mutation or truncation of the PDGF-DD protein. An agonist of the PDGF-DD protein can retain substantially the same, or a subset of, the biological activities of the naturally occurring form of the PDGF-DD protein. An antagonist of the PDGF-DD protein can inhibit one or more of the activities of the naturally occurring form of the PDGF-DD protein by, for example, competitively binding to a downstream or upstream member of a cellular signaling cascade which includes the PDGF-DD protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the PDGF-DD protein.

Variants of the PDGF-DD protein that function as either PDGF-DD agonists (mimetics) or as PDGF-DD antagonists can be identified by screening combinatorial libraries of mutants, e.g., truncation mutants, of the PDGF-DD protein for protein agonist or antagonist activity. In one embodiment, a variegated library of PDGF-D variants is generated by combinatorial mutagenesis at the nucleic acid level and is encoded by a variegated gene library. A variegated library of PDGF-D variants can be produced by, for example, enzymatically ligating a mixture of synthetic oligonucleotides into gene sequences such that a degenerate set of potential PDGF-D sequences is expressible as individual polypeptides, or alternatively, as a set of larger fusion proteins (e.g., for phage display) containing the set of PDGF-D sequences therein. There are a variety of methods which can be used to produce libraries of potential PDGF-D variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential PDGF-D variant sequences. Methods for synthesizing degenerate oligonucleotides are known in the art (see, e.g., Narang, Tetrahedron 39:3 (1983); Itakura et al., Annu. Rev. Biochem. 53:323 (1984); Itakura et al., Science 198:1056 (1984); Ike et al., Nucl. Acid Res. 11:477 (1983).

Design and Generation of Other Therapeutics

Moreover, based on the activity of the antibodies that are produced and characterized herein with respect to PDGF-DD, the design of other therapeutic modalities beyond antibody moieties is facilitated. Such modalities include, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules.

In connection with the generation of advanced antibody therapeutics, where complement fixation is a desirable attribute, it may be possible to sidestep the dependence on complement for cell killing through the use of bispecifics, immunotoxins, or radiolabels, for example.

For example, in connection with bispecific antibodies, bispecific antibodies can be generated that comprise (i) two antibodies one with a specificity to PDGF-DD and another to a second molecule that are conjugated together, (ii) a single antibody that has one chain specific to PDGF-DD and a second chain specific to a second molecule, or (iii) a single chain antibody that has specificity to PDGF-DD and the other molecule. Such bispecific antibodies can be generated using techniques that are well known for example, in connection with (i) and (ii) see, e.g., Fanger et al., Immunol Methods 4:72-81 (1994) and Wright and Harris, supra and in connection with (iii) see, e.g., Traunecker et al., Int. J. Cancer (Suppl.) 7:51-52 (1992). In each case, the second specificity can be made to the heavy chain activation receptors, including, without limitation, CD16 or CD64 (see, e.g., Deo et al., 18:127 (1997)) or CD89 (see, e.g., Valerius et al., Blood 90:4485-4492 (1997)). Bispecific antibodies prepared in accordance with the foregoing would be likely to kill cells expressing PDGF-DD, and particularly those cells in which the PDGF-DD antibodies described herein are effective.

With respect to immunotoxins, antibodies can be modified to act as immunotoxins utilizing techniques that are well known in the art. See, e.g., Vitetta, Immunol Today 14:252 (1993). See also U.S. Pat. No. 5,194,594. In connection with the preparation of radiolabeled antibodies, such modified antibodies can also be readily prepared utilizing techniques that are well known in the art. See, e.g., Junghans et al., in Cancer Chemotherapy and Biotherapy 655-686 (2d ed., Chafner and Longo, eds., Lippincott Raven (1996)). See also U.S. Pat. Nos. 4,681,581, 4,735,210, 5,101,827, 5,102,990 (RE 35,500), 5,648,471, and 5,697,902. Each of immunotoxins and radiolabeled molecules would be likely to kill cells expressing PDGF-DD, and particularly those cells in which the antibodies described herein are effective.

In connection with the generation of therapeutic peptides, through the utilization of structural information related to PDGF-DD and antibodies thereto, such as the antibodies described herein (as discussed below in connection with small molecules) or screening of peptide libraries, therapeutic peptides can be generated that are directed against PDGF-DD. Design and screening of peptide therapeutics is discussed in connection with Houghten et al., Biotechniques 13:412-421 (1992), Houghten, PNAS USA 82:5131-5135 (1985), Pinalla et al., Biotechniques 13:901-905 (1992), Blake and Litzi-Davis, BioConjugate Chem. 3:510-513 (1992). Immunotoxins and radiolabeled molecules can also be prepared, and in a similar manner, in connection with peptidic moieties as discussed above in connection with antibodies.

Assuming that the PDGF-DD molecule (or a form, such as a splice variant or alternate form) is functionally active in a disease process, it will also be possible to design gene and antisense therapeutics thereto through conventional techniques. Such modalities can be utilized for modulating the function of PDGF-DD. In connection therewith the antibodies, as described herein, facilitate design and use of functional assays related thereto. A design and strategy for antisense therapeutics is discussed in detail in International Patent Application No. WO 94/29444. Design and strategies for gene therapy are well known. However, in particular, the use of gene therapeutic techniques involving intrabodies could prove to be particularly advantageous. See, e.g., Chen et al., Human Gene Therapy 5:595-601 (1994) and Marasco, Gene Therapy 4:11-15 (1997). General design of and considerations related to gene therapeutics is also discussed in International Patent Application No.: WO 97/38137.

Small molecule therapeutics can also be envisioned. Drugs can be designed to modulate the activity of PDGF-DD, as described herein. Knowledge gleaned from the structure of the PDGF-DD molecule and its interactions with other molecules, as described herein, such as the antibodies described herein, and others can be utilized to rationally design additional therapeutic modalities. In this regard, rational drug design techniques such as X-ray crystallography, computer-aided (or assisted) molecular modeling (CAMM), quantitative or qualitative structure-activity relationship (QSAR), and similar technologies can be utilized to focus drug discovery efforts. Rational design allows prediction of protein or synthetic structures which can interact with the molecule or specific forms thereof which can be used to modify or modulate the activity of PDGF-DD. Such structures can be synthesized chemically or expressed in biological systems. This approach has been reviewed in Capsey et al., Genetically Engineered Human Therapeutic Drugs (Stockton Press, NY (1988)). Further, combinatorial libraries can be designed and synthesized and used in screening programs, such as high throughput screening efforts.

Therapeutic Administration and Formulations

The anti-PDGF-DD compounds including, but not limited to, antibodies and fragments thereof are suitable for incorporation into pharmaceuticals that treat organisms in need of a compound that modulates PDGF-DD. These pharmacologically active compounds can be processed in accordance with conventional methods of galenic pharmacy to produce medicinal agents for administration to organisms, e.g., animals and mammals including humans. In certain embodiments, the active ingredients can be incorporated into a pharmaceutical product with or without modification. Additional embodiments include the manufacture of pharmaceuticals or therapeutic agents that deliver the pharmacologically active compounds, described herein, by several routes. For example, and not by way of limitation, DNA, RNA, and viral vectors having sequence encoding the antibodies or fragments thereof can be used in certain embodiments. Additionally, nucleic acids encoding antibodies or fragments thereof can be administered alone or in combination with other active ingredients.

It will be appreciated that administration of therapeutic entities described herein can be administered in admixture with suitable carriers, excipients, stabilizers, and other agents that are incorporated into formulations to provide improved transfer, delivery, tolerance, and the like. Pharmaceutically acceptable carriers include organic or inorganic carrier substances suitable for parenteral, enteral (for example, oral) or topical application that do not deleteriously react with the pharmacologically active ingredients of this invention. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Additional carriers, excipients, and stabilizers include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol. Many more suitable vehicles are described in Remmington's Pharmaceutical Sciences, 15th Edition, Easton:Mack Publishing Company, pages 1405-1412 and 1461-1487(1975) and The National Formulary XIV, 14th Edition, Washington, American Pharmaceutical Association (1975), herein incorporated by reference.

The route of antibody administration can be in accord with known methods, including, for example, but are not limited to, topical, transdermal, parenteral, gastrointestinal, transbronchial, and transalveolar. Parenteral routes of administration include, but are not limited to, electrical or direct injection or infusion such as direct injection into a central venous line, intravenous, intracerebral, intramuscular, intraperitoneal, intradermal, intraarterial, intrathecal, or intralesional routes. The antibody is preferably administered continuously by infusion, by bolus injection, or by sustained release systems as noted below. In a preferred embodiment the administration route can be subcutaneous injection. In an alternative embodiment, the antibodies are administered via the renal artery. Gastrointestinal routes of administration include, but are not limited to, ingestion and rectal. Transbronchial and transalveolar routes of administration include, but are not limited to, inhalation, either via the mouth or intranasally.

When used for in vivo administration, the antibody formulation may be sterile. This can be readily accomplished by filtration through sterile filtration membranes, prior to or following lyophilization and reconstitution. The antibody ordinarily will be stored in lyophilized form or in solution. In addition, the therapeutic composition can be pyrogen-free and in a parenterally acceptable solution having due regard for pH, isotonicity, and stability. Therapeutic antibody compositions generally are placed into a container having a sterile access port, for example, an intravenous solution bag or vial having a stopper pierceable by a hypodermic injection needle.

Sterile compositions for injection can be formulated according to conventional pharmaceutical practice as described in Remington's Pharmaceutical Sciences (18^{th} ed., Mack Publishing Company, Easton, Pa. (1990)). The pharmaceutical preparations can be sterilized and if desired mixed with auxiliary agents, for example, lubricants, preservatives, stabilizers, wetting agents, emulsifiers, salts for influencing osmotic pressure, buffers, antioxidants, coloring, flavoring and/or aromatic substances and the like that do not deleteriously react with the active compounds. For example, dissolution or suspension of the active compound in a vehicle such as water or naturally occurring vegetable oil like sesame, peanut, or cottonseed oil or a synthetic fatty vehicle like ethyl oleate or the like may be desired.

Suitable compositions having the pharmacologically active compounds of this invention that are suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, intradermal, or subcutaneous injection.

Compositions having the pharmacologically active compounds of this invention that are suitable for gastrointestinal administration include, but not limited to, pharmaceutically acceptable powders, pills or liquids for ingestion and suppositories for rectal administration.

Suitable examples of sustained-release preparations include semipermeable matrices of solid hydrophobic polymers containing the polypeptide, which matrices are in the form of shaped articles, films or microcapsules. Examples of sustained-release matrices include polyesters, hydrogels (e.g., poly(2-hydroxyethyl-methacrylate) as described by Langer et al., J Biomed Mater. Res., 15:167-277 (1981) and Langer, Chem. Tech., 12:98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22:547-556 (1983)), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through disulfide interchange, stabilization may be achieved by modifying sulfhydryl residues, lyophilizing from acidic solutions, controlling moisture content, using appropriate additives, and developing specific polymer matrix compositions.

Sustained-release compositions also include liposomally entrapped antibodies of the invention. Liposomes containing such antibodies are prepared by methods known per se: U.S. Pat. No. DE 3,218,121; Epstein et al., Proc. Natl. Acad. Sci. USA, 82:3688-3692 (1985); Hwang et al., Proc. Natl. Acad. Sci. USA, 77:4030-4034 (1980); EP 52,322; EP 36,676; EP 88,046; EP 143,949; 142,641; Japanese patent application 83-118008; U.S. Pat. Nos. 4,485,045 and 4,544,545; and EP 102,324.

An effective amount of antibody to be employed therapeutically will depend, for example, upon the therapeutic objectives, the route of administration, and the condition of the patient. The dosage of the antibody will be determined by the attending physician taking into consideration various factors known to modify the action of drugs including severity and type of disease, body weight, sex, diet, time and route of administration, other medications and other relevant clinical factors. Accordingly, it will be necessary for the therapist to titer the dosage and modify the route of administration as required to obtain the optimal therapeutic effect. Typically, the clinician will administer antibody until a dosage is reached that achieves the desired effect. Therapeutically effective dosages may be determined by either in vitro or in vivo methods. The progress of this therapy is easily monitored by conventional assays or by the assays described herein.

Therapeutic efficacy and toxicity of such compounds can be determined by standard pharmaceutical procedures in cell cultures or experimental animals, for example, ED50 (the dose therapeutically effective in 50% of the population). The data obtained from treating the rat model of nephritis or an alternative model may be used in formulating a range of dosage for use with other organisms, including humans. The dosage of such compounds lies preferably within a range of circulating concentrations that include the ED50 with no toxicity. The dosage varies within this range depending upon type of evectin, hybrid, binding partner, or fragment thereof, the dosage form employed, sensitivity of the organism, and the route of administration.

Normal dosage concentrations of various antibodies or fragments thereof can vary from approximately 0.1 to 100 mg/kg. Desirable dosage concentrations include, for example, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, and 100 mg/kg or more. One preferred dosage is 1 to 10 mg/kg.

In some embodiments, the dose of antibodies or fragments thereof produces a tissue or blood concentration or both from approximately 0.1 μM to 500 mM, preferably about 1 to 800 μM, and more preferably greater than about 10 μM to about 500 μM. Preferable doses are, for example, the amount required to achieve a tissue or blood concentration or both of 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 120 μM, 130 μM, 140 μM, 145 μM, 150 μM, 160 μM, 170 μM, 180 μM, 190 μM, 200 μM, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. In alternative embodiments, doses that produce a tissue concentration of greater than 800 μM are can be used. A constant infusion of the antibodies, hybrids, binding partners, or fragments thereof can also be provided so as to maintain a stable concentration in the tissues as measured by blood levels.

Dosage and administration can be adjusted to provide sufficient levels of the active moiety or to maintain the desired effect. Embodiments herein include both short acting and long acting pharmaceutical compositions. Accordingly, embodiments include schedules where pharmaceutical compositions are administered approximately every 1, 2, 3, 4, 5, or 6 days, every week, once every 2 weeks, once every 3 weeks, once every 4 weeks, once every 5 weeks, once every 6 weeks, once every 7 weeks, or once every 8 weeks. Depending on half-life and clearance rate of the particular formulation, the pharmaceutical compositions described herein can be administered about once, twice, three, four, five, six, seven, eight, nine, and ten or more times per day.

Additional therapeutics may be administered in combination with, before, or after administration of the anti-PDGF-DD antibodies. These therapeutics may be used to treat symptoms of the disease or may decrease the side effects of the anti-PDGF-DD antibodies. They may also be used to enhance the activity of the anti-PDGF-DD antibodies. Any type of therapeutic may be used including, but not limited to, for example, antibiotics, diuretics, anesthetics, analgesics, anti-inflammatories, and insulin. Examples of agents that are typically used to treat glomerulonephritis and may be used in combination with the antibodies include prednisone, cyclophosphamide, chlorambucil, and blood thinning agents, such as, for example, warfarin, dipyradamole, and aspirin.

Diagnostic Use

PDGF-DD has been found to be expressed at low levels in normal kidney but its expression is increased dramatically in postischemic kidney (Ichimura T, Bonventre J V, Bailly V, Wei H, Hession C A, Cate R L, Sanicola M., J. Biol. Chem. 273(7):4135-42 (1998)). As immunohistochemical staining with anti-PDGF-DD antibody shows positive staining of renal, kidney, prostate and ovarian carcinomas (see below), PDGF-DD overexpression relative to normal tissues can serve as a diagnostic marker of such diseases.

Accordingly, embodiments of the invention are also useful for assays, particularly in vitro diagnostic assays, for example, for use in determining the level of PDGF-DD in patient samples. Such assays may be useful for diagnosing diseases associated with over expression of PDGF-DD. In some embodiments, the disease is nephritis. The patient samples can be, for example, bodily fluids, preferably blood, more preferably blood serum, synoival fluid, tissue lysates, and extracts prepared from diseased tissues. Other embodiments of the invention are useful for diagnosing and staging nephritis and diseases related to mesangial proliferation. Monitoring the level of PDGF-DD may be used as a surrogate measure of patient response to treatment and as a method of monitoring the severity of the disease in a patient. Elevated levels of PDGF-DD compared to levels of other soluble markers would indicate the presence of postischemic kidney. The concentration of the PDGF-DD antigen present in patient samples can be determined using a method that specifically determines the amount of the antigen that is present. Such a method includes an ELISA method in which, for example, antibodies of the invention may be conveniently immobilized on an insoluble matrix, such as a polymer matrix. Alternatively, immunohistochemistry staining assays using anti-PDGF-DD antibodies may be used to determine levels of PDGF-DD in a sample. Using a population of samples that provides statistically significant results for each stage of progression or therapy, a range of concentrations of the antigen that may be considered characteristic of each stage of disease can be designated.

In one embodiment, a sample of blood is taken from the subject and the concentration of the PDGF-DD antigen present in the sample is determined to evaluate the stage of the disease in a subject under study, or to characterize the response of the subject to a course of therapy. The concentration so obtained is used to identify in which range of concentrations the value falls. The range so identified correlates with a stage of disease progression or a stage of therapy identified in the various populations of diagnosed subjects, thereby providing a stage in the subject under study.

Gene amplification and/or expression may be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay can be carried out where the duplex is bound to a surface, so that upon the formation of duplex on the surface, the presence of antibody bound to the duplex can be detected.

For example, antibodies, including antibody fragments, can be used to qualitatively or quantitatively detect the expression of PDGF-DD proteins. As noted above, the antibody preferably is equipped with a detectable, e.g., fluorescent label, and binding can be monitored by light microscopy, flow cytometry, fluorimetry, or other techniques known in the art. These techniques are particularly suitable if the amplified gene encodes a cell surface protein, e.g., a growth factor. Such binding assays are performed as known in the art.

In situ detection of antibody binding to the PDGF-DD protein can be performed, for example, by immunofluorescence or immunoelectron microscopy. For this purpose, a tissue specimen is removed from the patient, and a labeled antibody is applied to it, preferably by overlaying the antibody on a biological sample. This procedure also allows for determining the distribution of the marker gene product in the tissue examined. It will be apparent for those skilled in the art that a wide variety of histological methods are readily available for in situ detection.

One of the most sensitive and most flexible quantitative methods for quantitating differential gene expression is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from a disease tissue and corresponding normal tissues, respectively. Thus, mRNA can be extracted, for example, from frozen or archived paraffin-embedded and fixed (e.g. formalin-fixed) samples of diseased tissue for comparison with normal tissue of the same type. Methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest., 56:A67 (1987), and De Andres et al., BioTechniques, 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Total RNA from tissue samples can be isolated using RNA Stat-60 (Tel-Test).

As RNA cannot serve as a template for PCR, the first step in differential gene expression analysis by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ endonuclease activity. Thus, TaqMan PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicontypical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan RT-PCR can be performed using commercially available equipments, such as, for example, ABI PRIZM 7700TM Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRIZM 7700TM Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct). The ΔCt values are used as quantitative measurement of the relative number of starting copies of a particular target sequence in a nucleic acid sample when comparing the expression of RNA in a cell from a diseased tissue with that from a normal cell.

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

Differential gene expression can also be identified, or confirmed using the microarray technique. In this method, nucleotide sequences of interest are plated, or arrayed, on a microchip substrate. The arrayed sequences are then hybridized with specific DNA probes from cells or tissues of interest.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip selectively hybridize to each spot of DNA on the array. After stringent washing to remove non-specifically bound probes, the chip is scanned by confocal laser microscopy. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA, 93(20)L106-49). The methodology of hybridization of nucleic acids and microarray technology is well known in the art.

Selected embodiments of the antibodies and methods are illustrated in the Examples below:

### EXAMPLES

The following examples, including the experiments conducted and results achieved are provided for illustrative purposes only and are not to be construed as limiting upon the embodiments of the invention described herein.

### Example 1

### PDGF-DD Antigen Preparation

Recombinant human and murine PDGF-DD lacking the CUB-domain, that is biologically active PDGF-DD p35, was produced as described in LaRochelle et al., Nat Cell Biol 3:517-521 (2001). Human PDGF-CC was produced by the same protocol. PDGF-AA and PDGF-BB were purchased from R & D Systems (Minneapolis, Minn.).

### Example 2

### Aptamer Based Antagonist Against PDGF

The synthesis and characterization of the PDGF-B aptamer (NX1975) has been described in detail. Green et al., Biochemistry 35:14413-14424 (1996). Modifications of the original DNA aptamer involved substitutions of certain nucleotides with 2-fluoropyrimidines and 2′-O-methylpurines to improve nuclease resistance as well as coupling of the aptamer to 40 kDa polyethylene glycol (PEG) to prolong its plasma residence time in vivo. Floege et al., Am J. Pathol 154:169-179 (1999).

### Example 3

### Anti-PDGF-DD Antibodies

Fully human anti-PDGF-DD monoclonal antibodies were generated as described in Yang et al., J. Leukoc. Biol. 66:401-410 (1999), with the following modifications. Briefly, the human IgG2 bearing XenoMouse® strain (8-10 weeks old) was immunized twice weekly by footpad injection with 10 μg of V5-tagged soluble PDGF-DD, LaRochelle et al., Nat Cell Biol 3:517-521 (2001), in complete Freund's adjuvant. Yang et al., supra. Hybridomas were generated utilizing electro-cell fusion. Fully human isotype matched PK16.3 was used as the negative control.

### Example 4

### Characterization of the Fully Human Anti-PDGF-DD mAB 6.4

The specificity of fully human anti-PDGF-DD mAb 6.4 for PDGF-DD among the PDGFs was characterized by solid phase ELISA, western blot analysis, and NIH 3T3 BrdU incorporation analysis.

Solid Phase ELISA

The specificity of the fully human anti-PDGF-DD was characterized by solid-phase ELISA. Briefly, Corning 96-well flat bottom high protein binding polystyrene microtiter plates were coated with 500 ng/ml PDGF-AA, PDGF-BB, PDGF-CC, or PDGF-DD overnight. Plates were blocked with Assay Diluent (Pharmingen, San Diego, Calif.) for 1 hour. Anti-PDGF-DD mAb 6.4 or control mAb PK16.3 was then added at the indicated concentration for 2 hours. Primary mAb binding was detected using anti-human horseradish peroxidase conjugated secondary antibody with TMB Reagent (Pharmingen, San Diego, Calif.). Microtiter plates were read at 450 nm with a Kinetic Microplate Reader (Molecular Devices, Sunnyvale, Calif.).

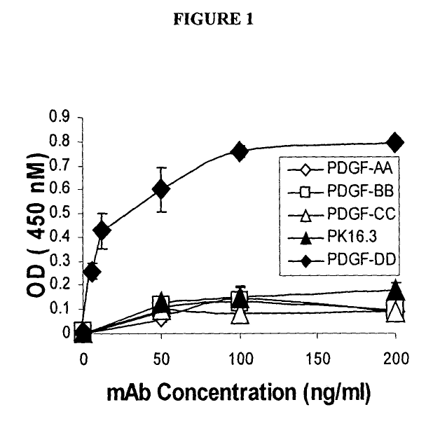

As shown in FIG. 1, anti-PDGF-DD mAb 6.4 recognized PDGF-DD, but not PDGF-AA, PDGF-BB or PDGF-CC. Control mAb PK16.3 showed no recognition of PDGF-DD. To confirm the ELISA result, western blot analysis was also performed.

Additionally, PDGF solid-phase ELISA was performed by coating Corning 96-well flat-bottom high-protein binding polystyrene microtiter plates with 500 ng/ml human or murine PDGF-DD overnight. Plates were blocked with Assay Diluent (Pharmingen, San Diego, Calif.) for 1 hour. Anti-PDGF-DD mAb 6.4 was then added at the indicated concentration for 2 hours. Primary mAb binding was detected using anti-human horse-radish peroxidase-conjugated secondary antibody with TMB reagent (Pharmingen). Microtiter plates were read at 450 nm with a Kinetic Microplate Reader (Molecular Devices, Menlo Park, Calif.).

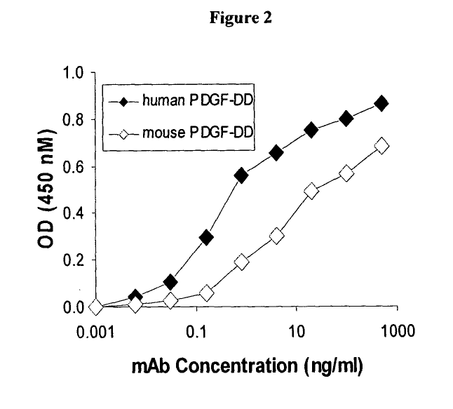

As shown in the FIG. 2, anti-PDGF-DD mAb 6.4 antibody recognizes both human and murine PDGF-DD.

Western Blot Analysis

For western blot analysis, PDGF-AA, PDGF-BB, PDGF-CC and PDGF-DD (250 ng) were diluted in SDS-PAGE sample buffer, boiled and subjected to SDS-PAGE gel electrophoresis using a 16% SDS-polyacrylamide gels. Proteins were transferred to Hybond-P membranes (Amersham, Piscataway, N.J.) and filters were probed with PDGF-DD mAb 6.4 or control mAb PK16.3 (0.85 μg/ml) for 12 hours. After washing, filters were incubated with anti-human horseradish peroxidase conjugated secondary antibody. Bands were visualized by enhanced chemiluminescence (Amersham, Piscataway, N.J.).

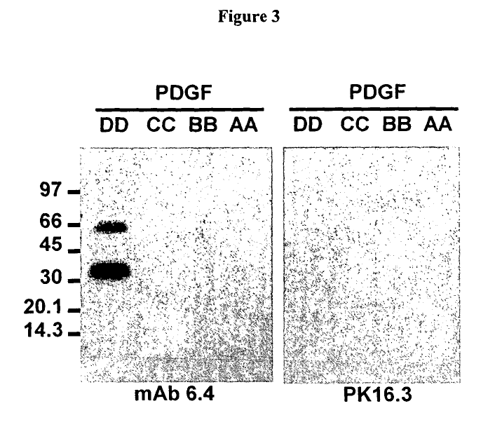

FIG. 3, shows that anti-PDGF-DD mAb 6.4 immunoblotted PDGF-DD, p35, but not PDGF-AA, PDGF-BB or PDGF-CC. Control mAb PK16.3 recognized no PDGFs. BIACor kinetic measurements were used to determine that the affinity of anti-PDGF-DD antibody 6.4 for human PDGF-DD was 170 pM and anti-PDGF-DD mAb 6.4 had at least a 20-fold lower affinity for murine PDGF-DD (data not shown). NIH 3T3 BrdU Incorporation Assay

To test the ability of anti-PDGF-DD mAb 6.4 to neutralize PDGF-DD-induced mitogenic activity, a NIH 3T3 BrdU incorporation assay was used. The NIH 3T3 neutralization assay was performed as described in LaRochelle et al., Nat Cell Biol 3:517-521 (2001), with the following modifications. Briefly, NIH 3T3 cells were serum starved for 24 hours and monoclonal antibody added at the indicated concentration. PDGF-DD was then added at 100 ng/ml. After 18 hrs, BrdU was added for 5 hrs and the BrdU assay performed according to the manufacturer's specifications (Roche).

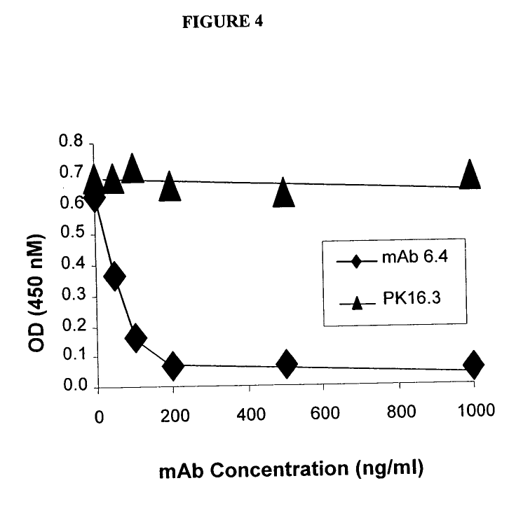

As shown in FIG. 4, anti-PDGF-DD mAb 6.4 neutralized PDGF-DD-induced BrdU incorporation with an IC₅₀ of approximately 75 ng/ml. PDGF-BB-induced BrdU incorporation was not affected at the highest concentrations tested (5 μg/ml, data not shown). Control mAb PK16.3 did not affect PDGF-DD-induced BrdU incorporation. Taken together, these results demonstrate that anti-PDGF-DD mAb 6.4 is highly specific for PDGF-DD, does not recognize other PDGF family members and potently neutralizes PDGF-DD-induced BrdU incorporation.

### Example 5

### Effect of PDGF-DD on Mesangial Cell Proliferation In Vitro

Mesangial Cell Culture Experiments

To study the effects of PDGF-DD on mesangial cell proliferation in vitro, Rat mesangial cells were established in culture, characterized and maintained as described previously. Radeke et al., J Immunol 153:1281-1292, (1994). Briefly, rat mesangial cells were seeded in 96-well plates (Nunc, Wiesbaden, Germany), grown to subconfluency and growth-arrested for 48 hours in RPMI 1640 with 1% bovine serum albumin. After 48 hours, PDGF-DD (10-200 ng/ml) and PDGF-BB (10 ng/ml and 50 ng/ml) together with PDGF-B-chain aptamer (10 ng/ml) or sequence-scrambled aptamer (100 ng/ml) were added and the cells were incubated for 24 hours. DNA synthesis was determined by BrdU incorporation and measured by a calorimetric cell proliferation ELISA (Roche, Mannheim, Germany) according to the instructions of the manufacturer.

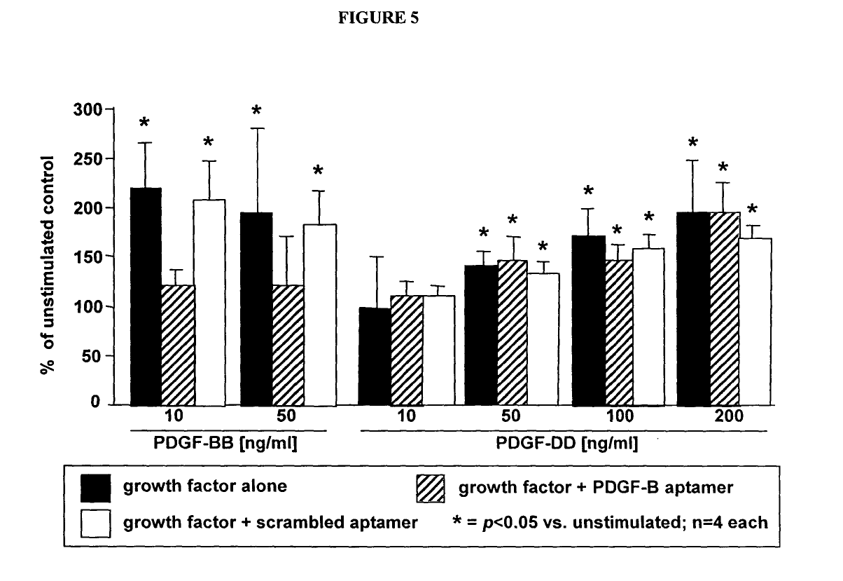

Incubation of growth-arrested cultured rat mesangial cells with PDGF-DD led to a dose-dependent increase in proliferation (FIG. 5). Data are means ±SD of four independent experiments. Statistical significance (defined as p<0.05) was evaluated using ANOVA and Bonferroni t-tests. * indicates p<0.05 versus unstimulated control.

Independence of the mitogenic PDGF-DD activity from PDGF-B was demonstrated by incubating the cells with antagonistic PDGF-B aptamers or sequence-scrambled control aptamers simultaneously to PDGF-DD. While the aptamers blocked PDGF-BB induced proliferation, they had no effect on the mitogenic potential of PDGF-DD (FIG. 5). Similar data were obtained with human mesangial cells (not shown).

### Example 6

### Effect of PDGF-DD and Anti-PDGF-DD Antibodies on Human Mesangial Cells (HMC)

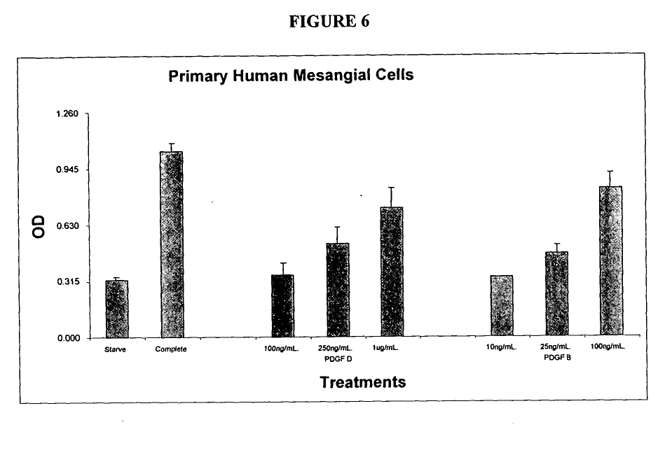

Human Mesangial cells were serum starved and treated overnight with BrdU along with PDGF-DD or PDGF-BB at the following concentrations 100 ng/mL, 250 ng/mL, 11 g/mL. For comparison, other mesenchymal cells, for example, NIH 3T3 fibroblasts, CCD 1070 foreskin fibroblasts, and primary smooth-muscle cells, were treated with BrdU and complete serum. BrdU incorporation was detected by assay with an anti-BrdU antibody ELISA. As FIG. 6 demonstrates, PDGF-DD was found to induce the proliferation of primary human mesangial cells at concentrations above 100 ng/mL. FIG. 6 further illustrates that a ten-fold difference was noted in the concentrations of PDGF-DD and PDGF-BB that was required for similar induction of BrdU incorporation on human mesangial cells.

### Example 7

### PDGF-DD Levels in Nephritic Sera

A sandwich ELISA was developed to quantify PDGF-DD levels in human serum. The two fully human mAbs (anti-PDGF-DD mAbs 1.6 and 1.17) used in the sandwich ELISA recognized different epitopes on the PDGF-DD molecule (data not shown). Anti-PDGF-DD mAb 1.6 was used as the capture antibody, and anti-PDGF-DD mAb 1.17 was used as the detection antibody.

The ELISA was performed as follows: 50 μl of capture antibody (anti-PDGF-DD mAb 1.6) in coating buffer (0.1 M NaHCO₃, pH 9.6) at a concentration of 2 μg/ml was coated on ELISA plates (Fisher). After incubation at 4° C. overnight, the plates were treated with 200 μl of blocking buffer (0.5% BSA, 0.1% Tween 20, 0.01% Thimerosal in PBS) for 1 hour at 25° C. The plates were washed (3×) using 0.05% Tween 20 in PBS (washing buffer, WB). Normal or patient sera (Clinomics, Bioreclamation, Cooperative Human Tissue Network) were diluted in blocking buffer containing 50% human serum. The plates were incubated with serum samples overnight at 4° C., washed with WB, and then incubated with 100 μl/well of biotinylated detection anti-PDGF-DD mAb 1.17 for 1 hour at 25° C. After washing, the plates were incubated with HRP-streptavidin for 15 min, washed as before, and then treated with 100 μl/well of o-phenylenediamine in H₂O₂ (Sigma developing solution) for color generation. The reaction was stopped with 2M H₂SO₄ and analyzed using an ELISA plate reader at 492 nm. The concentration of PDGF-DD in serum samples was calculated by comparison to a PDGF-DD standard curve using a four-parameter curve fitting program.

PDGF-DD Serum Levels in Type II Diabetic Patients with Nephritis

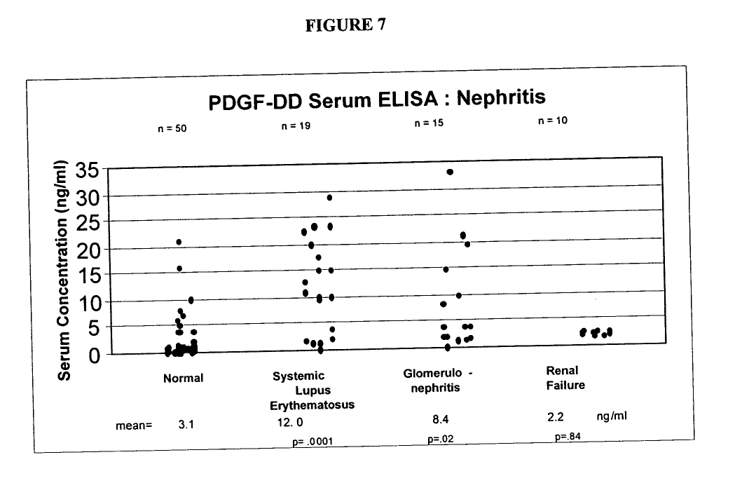

To determine whether PDGF-DD might be involved in nephritis, serum levels from patients with various types of nephritis, including type II diabetics were surveyed. Serum PDGF-DD concentrations were assessed using the quantitative PDGF-DD sandwich ELISA described above. The ELISA was specific for PDGF-DD and had a sensitivity of 4 ng/ml. FIG. 7 summarizes the results of the study. A closed circle represents the PDGF-DD concentration for an individual clinical serum sample. PDGF-DD serum concentrations are grouped according to the patient disease indication. The number of patients (n) for a given clinical indication is provided, along with the mean PDGF-DD concentration in ng/ml.

As shown in FIG. 7, PDGF-DD was elevated (mean=11.4 ng/ml p<0.001) in 8 of 10 serum samples from patients with type II diabetes compared to 6% of normal sera (n=50). The mean serum levels of PDGF-DD in type II diabetes patients ranged from around 4 to 24 ng/ml, compared to a concentration of less than 4 ng/ml in normal individuals. These data demonstrate that PDGF-DD is elevated in the sera of patients with type II diabetes suggesting that PDGF-DD may be a target to delay the onset of kidney disease/renal failure associated with type II diabetes. These results demonstrate that PDGF-DD levels are elevated four- to seven-fold in the sera of nephritis patients compared to the sera of normal individuals.

### Example 8

### Immunohistochemical Analysis of Rat Mesangium

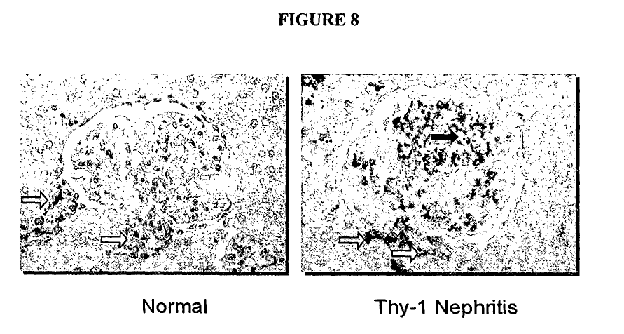

Normal rat mesangium cells were compared with the mesangium cells of rats with anti-Thy-1 induced nephritis. Wistar rats were obtained from Charles River. Immunohistochemical staining was performed with anti-PDGF-DD sera followed by detection with goat anti-rabbit conjugated to horseradish peroxidase. Briefly, tissues were deparaffinized using conventional techniques, and treated with trypsin (0.15%) for 10 minutes at 37° C. After incubation with primary antibody and anti-rabbit-HRP conjugate for 10 minutes each, a solution of diaminobenzidine (DAB) was applied onto the sections to visualize the immunoreactivity. As shown in FIG. 8, immunohistochemical analysis revealed elevated anti-PDGF-DD levels in rats with anti-Thy-1 induced nephritis. Mesangium, tubules and surrounding vasculature is shown. Mesangium cells included pericytes and renal tubules. White and gray arrows depict capillary and tubule staining respectively.

### Example 9

### Simulated Pharmacokinetics of a Fully Human Anti-PDGF-DD mAb 6.4

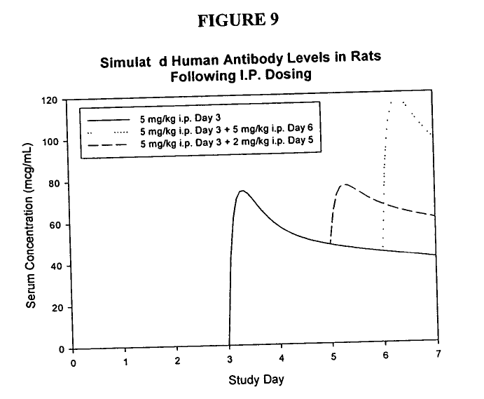

Simulated fully human mAb kinetics in rats was performed. Male Wistar rats were dosed with 10 mg/kg and 5 mg/kg of anti-PDGF-DD mAb 6.4 on day 3 and day 5, respectively. Sera were harvested and human anti-PDGF-DD mAb levels were quantitated using a human-specific IgG ELISA. As indicated in FIG. 9, there was not much peak to trough fluctuation over 4 days, even after a single dose. These data correlated favorably with the pK simulated model of human antibody clearance in rats, indicating that much of the anti-PDGF-DD mAb 6.4 remained in circulation once administered.

In an additional experiment to analyze antibody clearance rates, forty-nine (49) rats were treated with varying levels of anti-PDGF-DD antibodies, control antibodies, or PBS, as described below.

| | | |
|---|---|---|
| Group A | animal # 1-10 | 5 mg/kg anti-PDGF-DD antibodies |
| Group B | animal # 11-20 | 10 mg/kg anti-PDGF-DD antibodies |
| Group C | animal # 21-30 | 20 mg/kg anti-PDGF-DD antibodies |
| Group D | animal # 31-40 | 20 mg/kg irrelevant control Ab |
| Group E | animal # 41-49 | PBS |

In the following table, under “Circulating antibody,” the left column shows the day 5 results for the 49 animals and the right column shows the day 8 sample for the corresponding animal.

TABLE 2
Anti-PDGF-DD Antibody Clearance

| | | Circulating | |
|---|---|---|---|
| | | antibody (μg/ml) | |
| Animal ID# | Group | Day 5 | Day 8 |
| 1 | 5 mg/kg anti-PDGF-DD mAb | 0.1 | 28.4 |
| 2 | 5 mg/kg anti-PDGF-DD mAb | 37.3 | 9.4 |
| 3 | 5 mg/kg anti-PDGF-DD mAb | <0.02 | 41.7 |
| 4 | 5 mg/kg anti-PDGF-DD mAb | 55.0 | 80.2 |
| 5 | 5 mg/kg anti-PDGF-DD mAb | 46.3 | 12.6 |
| 6 | 5 mg/kg anti-PDGF-DD mAb | <0.02 | 30.0 |
| 7 | 5 mg/kg anti-PDGF-DD mAb | 30.4 | 32.7 |
| 8 | 5 mg/kg anti-PDGF-DD mAb | 32.7 | 32.5 |
| 9 | 5 mg/kg anti-PDGF-DD mAb | 50.5 | 42.7 |
| 10 | 5 mg/kg anti-PDGF-DD mAb | 44.0 | 64.3 |
| 11 | 10 mg/kg anti-PDGF-DD mAb | 50.3 | 92.9 |
| 12 | 10 mg/kg anti-PDGF-DD mAb | 127.2 | 69.5 |
| 13 | 10 mg/kg anti-PDGF-DD mAb | 68.1 | 77.8 |
| 14 | 10 mg/kg anti-PDGF-DD mAb | 58.2 | 119.0 |
| 15 | 10 mg/kg anti-PDGF-DD mAb | 89.0 | 13.5 |
| 16 | 10 mg/kg anti-PDGF-DD mAb | <0.02 | 12.1 |
| 17 | 10 mg/kg anti-PDGF-DD mAb | 0.1 | 160.4 |
| 18 | 10 mg/kg anti-PDGF-DD mAb | 115.6 | 51.9 |
| 19 | 10 mg/kg anti-PDGF-DD mAb | 86.0 | 31.4 |
| 20 | 10 mg/kg anti-PDGF-DD mAb | 44.7 | 48.2 |
| 21 | 20 mg/kg anti-PDGF-DD mAb | 46.0 | 40.1 |
| 22 | 20 mg/kg anti-PDGF-DD mAb | 253.6 | 73.9 |
| 23 | 20 mg/kg anti-PDGF-DD mAb | 256.1 | 93.8 |
| 24 | 20 mg/kg anti-PDGF-DD mAb | 309.9 | 254.0 |
| 25 | 20 mg/kg anti-PDGF-DD mAb | 201.3 | 171.7 |
| 26 | 20 mg/kg anti-PDGF-DD mAb | 0.3 | 15.0 |
| 27 | 20 mg/kg anti-PDGF-DD mAb | 112.8 | 84.8 |
| 28 | 20 mg/kg anti-PDGF-DD mAb | 187.9 | 66.8 |
| 29 | 20 mg/kg anti-PDGF-DD mAb | 154.0 | 191.2 |
| 30 | 20 mg/kg anti-PDGF-DD mAb | 186.7 | 94.8 |
| 31 | 20 mg/kg irrelevant control Ab | 104.2 | 49.1 |
| 32 | 20 mg/kg irrelevant control Ab | 0.4 | 10.8 |
| 33 | 20 mg/kg irrelevant control Ab | 117.0 | 91.7 |
| 34 | 20 mg/kg irrelevant control Ab | 150.5 | 154.1 |
| 35 | 20 mg/kg irrelevant control Ab | 149.9 | 124.7 |
| 36 | 20 mg/kg irrelevant control Ab | 162.2 | 156.2 |
| 37 | 20 mg/kg irrelevant control Ab | 116.3 | 95.1 |
| 38 | 20 mg/kg irrelevant control Ab | 176.2 | 49.9 |
| 39 | 20 mg/kg irrelevant control Ab | 97.8 | 39.4 |
| 40 | 20 mg/kg irrelevant control Ab | 0.1 | <0.02 |
| 41 | PBS | <0.02 | <0.02 |
| 42 | PBS | <0.02 | <0.02 |
| 43 | PBS | <0.02 | <0.02 |
| 44 | PBS | <0.02 | <0.02 |
| 45 | PBS | <0.02 | <0.02 |
| 46 | PBS | 0.1 | <0.02 |
| 47 | PBS | <0.02 | <0.02 |
| 48 | PBS | <0.02 | <0.02 |
| 49 | PBS | 19.1 | <0.02 |

As shown in the above, table, the anti-PDGF-DD mAb 6.4 exhibited the expected circulating half-life as calculated in the pharmacokinetic models.

### Example 10

### PDGF-DD Expression in Glomeruli During Mesangioproliferative Nephritis

To study the kinetics of PDGF-DD expression in glomeruli during anti-Thy 1.1 nephritis, anti-Thy 1.1 mesangial proliferative glomerulonephritis was induced in male Wistar rats (Charles River, Sulzfeld, Germany) weighing 180 g by injection of 1 mg/kg monoclonal anti-Thy 1.1 antibody (clone OX-7; European Collection of Animal Cell Cultures, Salisbury, England). Forty-five (45) rats received the anti-Thy 1.1 antibody and were sacrificed at time points 4 h, day 1, 2, 4, 7, 9, 14, 21 and 28 after antibody injection (n=5 each). Following sacrifice, renal tissue as well as isolated glomeruli were obtained. Glomerular isolation was performed by differential sieving. Johnson et al., J Clin Invest 87:847-858 (1991). All glomerular isolates were checked microscopically and exhibited a purity of greater than 98%. In addition, adrenal tissue was obtained.

Glomerular RNA Extraction and Analyses

RNA was isolated from the glomeruli and the expression was measured by real time quantitative PCR. Briefly, total RNA was extracted from isolated rat glomeruli and adrenal gland with the guanidinium isothiocyanate/phenol/chloroform method using standard procedures. Chomczynski et al., Anal Biochem 162:156-159 (1987). The RNA content and the purity of the samples obtained was determined by UV spectrophotometry at 260 and 280 nm.

The cDNA syntheses were performed in a 30 μl reaction mix including 1 μg of total RNA, 1 μl of random-primer (6 nt, 250 ng/μl, Roche), 6 μl of M-MLV reverse transcriptase buffer (Invitrogen, Groningen, The Netherlands), 1.5 μl dNTP-mix (10 mM each, Amersham Pharmacia Biotech, Freiburg, Germany), 0.7 μl RNase-inhibitor (40 U/μl, Promega, Mannheim, Germany), 1 μl of M-MLV reverse trancriptase (200 U/μl, Invitrogen) and DEPC-treated H₂O. The mix was incubated for 10 minutes at 25° C. followed by 1 hour at 42° C.

Real time quantitative PCR was carried out using an ABI prism 7700 sequence detector (Applied Biosystems, Weiterstadt, Germany). In each reaction 0.7511 cDNA and 12.511 PCR Master Mix (Platinum Quantitative PCR SuperMix-UDG with ROX Reference Dye; Invitrogen) were used in a total of 25 μl volume. The PCR conditions were 50° C. for 2 minutes followed by 40 cycles of 95° C. for 15 seconds and 60° C. for 1 minute. Taqman primers and probes were designed from sequences in the Genbank database using the Primer Express software (Applied Biosystems). The sequences of primers and probes used in this study are listed in Table 3 below.

TABLE 3
Primers and Probes.

| | | | |
|---|---|---|---|
| Gene | Forward primer | Reverse primer | Taqman probe |

| | | | |
|---|---|---|---|
| Rat | 5′- | 5′- | 5′- |
| GAPDH | ACAAGATGGTGAAGG | AGAAGGCAGCCCTGG | CGGATTTGGCCGTA |
| | TCGGTG-3′ (SEQ ID | TAACC-3′ (SEQ ID | TCGGACGC-3′ (SEQ |
| | NO:83) | NO:84) | ID NO:85) |
| Rat | 5′- | 5′- | 5′- |
| PDGF-A | TTCTTGATCTGGCCCC | TTGACGCTGCTGGTGT | CAGTGCAGCGCTTC |
| | CAT-3′ (SEQ ID NO:86) | TACAG-3′ (SEQ ID | ACCTCCACA-3′ (SEQ |
| | | NO:87) | ID NO:88) |
| Rat | 5′- | 5′- | 5′- |
| PDGF-B | GCAAGACGCGTACAG | GAAGTTGGCATTGGTG | TCCAGATCTCGCGG |
| | AGGTG-3′ (SEQ ID | CGA-3′ (SEQ ID NO:90) | AACCTCATCG-3′ |
| | NO:89) | | (SEQ ID NO:91) |
| Rat | 5′- | 5′- | 5′- |
| PDGF-C | CAGCAAGTTGCAGCTC | GACAACTCTCTCATGC | CGACAAGGAGCAG |
| | TCCA-3′ (SEQ ID NO:92) | CGGG-3′ (SEQ ID NO:93) | AACGGAGTGCAA-3′ |
| | | | (SEQ ID NO:94) |
| Rat | 5′- | 5′- | 5′- |
| PDGF-D | ATCGGGACACTTTTGC | GTGCCTGTCACCCGAA | TTGCGCAATGCCAA |
| | GACT-3′ (SEQ ID NO:95) | TGTT-3′ (SEQ ID NO:96) | CCTCAGGAG-3′ (SEQ |
| | | | ID NO:97) |

PGDF-DD is Overexpressed in Glomeruli During Mesangioproliferative Nephritis

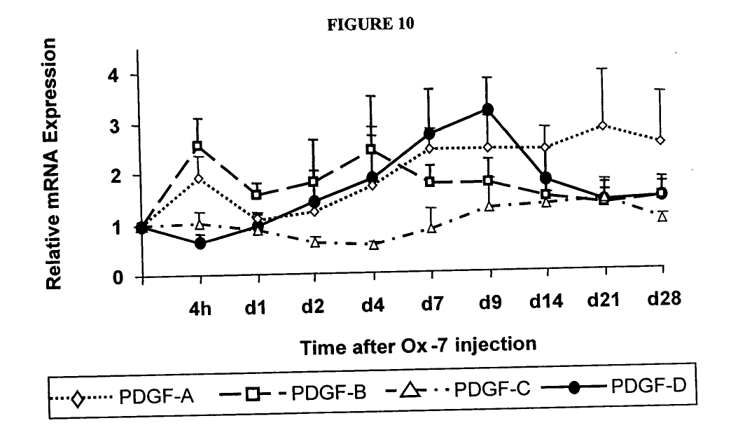

Following the induction of mesangioproliferative anti-Thy 1.1 nephritis in rats, glomerular PDGF-D mRNA expression initially decreased by 36% at 4 hours after disease induction, but then increased 2.4- to 2.9-fold between days 4 to 9 in comparison to non-nephritic rats (FIG. 10). This latter peak paralleled that of glomerular PDGF-A mRNA expression and occurred with some delay after the maximum PDGF-B mRNA expression (FIG. 10). In contrast to these three PDGF isoforms, PDGF-C mRNA was not upregulated during the first 28 days of anti-Thy 1.1 nephritis.

To assess whether PDGF-D mRNA upregulation during anti-Thy 1.1 nephritis is specific for the kidney, adrenal mRNA levels were also investigated, as the adrenal gland has been noted to be a prominent source of PDGF-D. LaRochelle et al., Nat Cell Biol 3:517-521 (2001). In contrast to glomeruli, no significant change in the PDGF-D mRNA expression level was observed in the adrenal glands during the first 28 days of anti-Thy 1.1 nephritis (data not shown). Despite these latter findings, a dramatic upregulation PDGF-DD protein levels was detected in the serum of nephritic rats on day 8 after disease induction (27.7±14.5 ng PDGF-D/ml, n=9) compared to the levels in normal animals which were consistently below the detection limit (<0.02 ng/ml, n=5).

Immunohistochemistry of PDGF-DD Expression

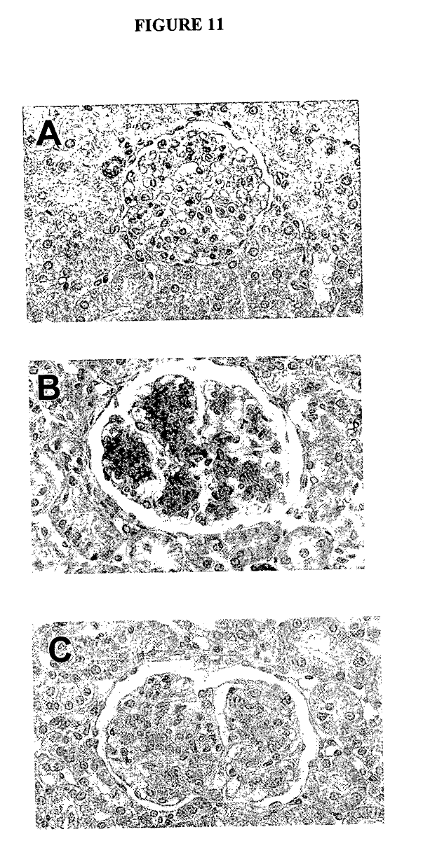

By immunohistochemistry PDGF-DD expression in normal rat kidney was confined to arterial and arteriolar vascular smooth muscle cells, whereas no immunoreactivity was noted in glomeruli (FIG. 11(A)). Prominent glomerular overexpression of PDGF-DD in the expanded mesangium was present at day 7 after disease induction (FIG. 11(B)), whereas the remaining staining pattern of the kidneys was not affected. No glomerular staining was present, when the anti-PDGF-DD antibody was replaced by an equal concentration of control IgG (FIG. 11(C)).

### Example 11

Interactions of PDGF-DD and PDGF-BB

Given that both PDGF-BB and PDGF-DD are overproduced in anti-Thy 1.1 nephritis (FIGS. 10 and 11) and given that antagonism of either results in a reduction of mesangioproliferative changes, potential interactions of the two PDGF isoforms were assessed.

Antagonism of PDGF-DD with anti-PDGF-DD mAb 6.4 had no significant effect on glomerular PDGF-B- and PDGF-D mRNA levels on day 8 of the disease (Table 11). Also, antagonism of PDGF-B by specific aptamers in this model led to no differences of the glomerular expression of PDGF-D mRNA on day 8 (3.18±0.58 increase over non-nephritic rats in the aptamer group versus 3.10±1.30 in the PEG40 control group, n=5 each). Glomerular PDGF-B mRNA expression in the latter experiment, however, was mildly induced by PDGF-B antagonism (3.31±1.1 in the aptamer group versus 2.52±0.64 in the PEG40 control group, n=5 each, expressions relative to those in normal rats). Measurements were performed twice for each sample.

### Example 12

### PDGF-DD Antagonism In Vivo

To study the effects of PDGF-DD antagonism in vivo, rats were treated with the anti-PDGF-DD antibody 6.4, control IgG PK16.3 or PBS on days 3 and 5 after induction of anti-Thy 1.1 nephritis. Treatment consisted of intraperitoneal injections of the antibodies dissolved in 80011 of 20 mM Tris-HC/100 mM NaCl, pH 7.4. Treatment timing was chosen to treat rats from about one day after onset to the peak of mesangial cell proliferation, which in the OX-7-induced anti-Thy 1.1 nephritis model occurs between days 5 and 8 after disease induction. The in vivo effects of three different dosages of the anti-PDGF-DD antibody were investigated.

The average dosage of 10 mg (day 3) plus 4 mg (day 5) anti-PDGF-DD mAb 6.4 per kg body weight was chosen based on calculations that this would result in serum levels of higher than 50 μg/ml, or half-maximal inhibition of PDGF-DD in vitro. To verify that relevant levels of anti-PDGF-DD mAb 6.4 or irrelevant control IgG2 PK16.3 were achieved, human IgG2 serum levels were measured in treatment groups 1-4 on days 5 and 8. Animals with levels below 30 μg/ml on day 5 were excluded from the analyses.

Altogether, seven groups of rats with sufficient human serum IgG2 in the antibody treated groups were studied: (1) Seven nephritic rats that received 5 mg/kg body weight of anti-PDGF-DD mAb 6.4 on day 3 and 2 mg/kg on day 5; (2) Seven nephritic rats that received 10 mg/kg body weight of anti-PDGF-DD mAb 6.4 on day 3 and 4 mg/kg on day 5; (3) Eight nephritic rats that received 20 mg/kg body weight of anti-PDGF-DD mAb 6.4 on day 3 and 8 mg/kg on day 5; (4) Eight nephritic rats that received 20 mg/kg body weight of irrelevant control IgG on day 3 and 8 mg/kg on day 5; (5) Nine nephritic rats that received equivalent injections of PBS alone; (6) Five non-nephritic, normal rats that received 10 mg/kg body weight of anti-PDGF-DD mAb 6.4 on day 3 and 4 mg/kg on day 5; and (7) Five non-nephritic, normal rats that received equivalent amounts of irrelevant control IgG.

In four randomly selected rats each from groups 1-5 renal biopsies for histological evaluation were obtained on day 5 by intravital biopsy as described. Floege et al., Am J Pathol 154:169-179 (1999). In all rats, post mortem biopsy was obtained on day 8 after disease induction. The remaining cortical tissue of 2 or 3 rats from every group was then pooled and used to isolate glomeruli (see above). Urine collections were performed on day 7 after disease induction. The thymidine analogue 5-bromo-2′-deoxyuridine (BrdU; Sigma, Deisenhofen, Germany; 100 mg/kg body weight) was injected intraperitoneal 4 hours prior to sacrifice on day 8.

### Inhibition of PDGF-DD In Vivo Reduces Pathological Mesangial Cell Proliferation

Following the injection of anti-Thy 1.1 antibody, PBS treated animals developed the typical course of the nephritis, which is characterized by early mesangiolysis and followed by a phase of mesangial cell proliferation and matrix accumulation on days 5 and 8. No obvious adverse effects were noted following the repeated injection of anti-PDGF-DD mAb 6.4 and all rats survived and appeared normal until the end of the study. Serum levels of the antibody that were achieved in the nephritic groups are shown in Table 4. Albumin/creatinine ratios in nephritic groups and systolic blood pressures in all treatment groups were not significantly different.

Urinary albumin levels were determined with an ELISA kit specific for rat albumin (Nephrat, Exocell, Philadelphia, Pa.). Urinary creatinine was determined by the method of two-point-kinetics with a Vitros 250 analyzer (Orthoclinical Diagnostics, Neckargmünd, Germany). All measurements were performed in duplicate. Blood pressure measurements were performed by the tail cuff method, using a programmed sphygmomanometer, BP-98A (Softron, Tokyo, Japan). Kitahara et al., J Am Soc Nephrol 13:1261-1270 (2002).

A considerable increase in albuminuria was present on day 7 in the nephritic as compared to non-nephritic rats (albumin/creatinine ratio: 15.5±5.6 mg/μmol in nephritic rats receiving PBS versus 0.3±0.1 mg/μmol in non-nephritic rats receiving control IgG; p<0.01). No significant differences were noted between the nephritic groups receiving either PBS, control IgG or the three dosages of anti-PDGF-DD mAb 6.4 (Table 4). Anti-PDGF-DD mAb 6.4 did not induce proteinuria in non-nephritic rats.

No significant effects of the various anti-PDGF-DD mAb 6.4 doses or of irrelevant control IgG on systemic blood pressure levels were observed and all animals remained normotensive on day 7 (Table 4).

TABLE 4
Human IgG2 antibody (anti-PDGF-DD mAb 6.4 or
irrelevant control IgG2) levels achieved in vivo,
urinary albumin/creatinine and systolic blood pressure

| | Human IgG2 serum | | Urinary | Systolic blood |
|---|---|---|---|---|
| | level [μg/ml] | | albumin/creatinine | pressure |
| | Day 5 after | Day 8 after | ratio [mg/μmol] | [mmHG] |
| | disease | disease | Day 7 after disease | Day 7 after |
| Groups | induction | induction | induction | disease induction |
| Nephritic + mAb 6.4 | 42 ± 9 | 39 ± 26 | 17.9 ± 9.4 | 112 ± 11 |
| 5 mg/kg (day 3) + 2 mg/kg | (n = 7) | (n = 7) | (n = 7) | (n = 3) |
| (day 5) | | | | |
| Nephritic + mAb 6.4 | 75 ± 29 | 65 ± 36 | 18.0 ± 6.7 | 136 ± 7 |
| 10 mg/kg (day 3) + 4 mg/kg | (n = 7) | (n = 7) | (n = 7) | (n = 3) |
| (day 5) | | | | |
| Nephritic + mAb 6.4 | 188 ± 85 | 112 ± 72 | 20.5 ± 23.3 | 131 ± 21 |
| 20 mg/kg (day 3) + 8 mg/kg | (n = 8) | (n = 8) | (n = 8) | (n = 4) |
| (day 5) | | | | |
| Nephritic + Control | 134 ± 29 | 95 ± 47 | 15.7 ± 4.7 | 119 ± 7 |
| IgG | (n = 8) | (n = 8) | (n = 8) | (n = 4) |
| 20 mg/kg (day 3) + 8 mg/kg | | | | |
| (day 5) | | | | |
| Nephritic + PBS | <0.02 (n = 9) | <0.02 (n = 9) | 15.5 ± 5.6 (n = 9) | 132 ± 15 (n = 5) |
| (day 3 and day 5) | | | | |
| Normal + mAb 6.4 | n.d. | n.d. | 0.2 ± 0.3 | 111 ± 11 |
| 10 mg/kg (day 3) + 4 mg/kg | | | (n = 5) | (n = 3) |
| (day 5) | | | | |
| Normal + Control IgG | n.d. | n.d. | 0.3 ± 0.1 | 122 ± 8 |
| 10 mg/kg (day 3) + 4 mg/kg | | | (n = 5) | (n = 3) |
| (day 5) | | | | |

** Data are mean values ± standard deviations.
n.d. = not determined.

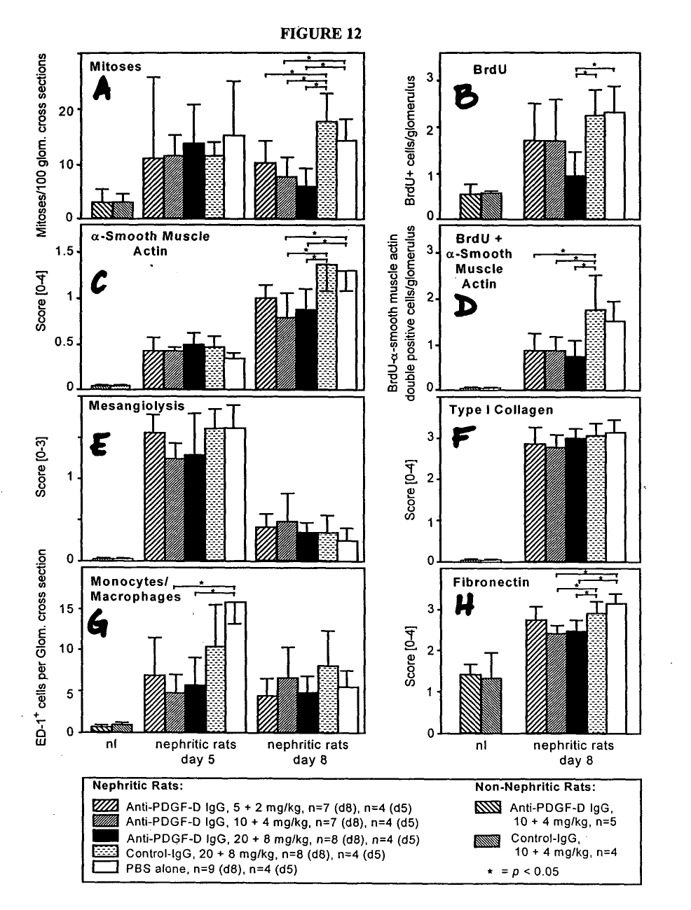

Glomerular cell proliferation, as assessed by counting the number of glomerular mitoses was significantly reduced in a dose dependent manner on day 8 in rats receiving the anti-PDGF-DD mAb 6.4 as compared to rats receiving irrelevant IgG or PBS (FIG. 12(A)). Treatment was carried out on days 3 and 5. Normal, or non-nephritic rats, were treated with anti PDGF-DD antibody or irrelevant control IgG. * indicates p<0.05. Counting of BrdU-positive nuclei confirmed these findings with the most pronounced suppression of proliferation on day 8 in the 20+8 mg anti-PDGF-DD antibody/kg treated group (FIG. 12(B)). When data of all three groups receiving anti-PDGF-DD treatment were pooled, the antibody levels achieved in vivo and BrdU-incorporating nuclei correlated negatively on day 5 (r=−0.53; p=0.018) and day 8 (r=−0.40; p=0.081).

To assess the treatment effects on mesangial cells, renal sections were immunostained for α-smooth muscle actin, which is expressed by activated mesangial cells only. Johnson et al., J Clin Invest 87:847-858 (1991). The glomerular expression of α-smooth muscle actin was significantly reduced on day 8 in the rats receiving 10+4 mg/kg and 20+8 mg/kg anti-PDGF-DD mAb 6.4 as compared to rats receiving irrelevant IgG or PBS (FIG. 12(C)). To specifically determine whether mesangial cell proliferation was reduced, anti-PDGF-DD mAb 6.4 treated rats and control IgG or PBS-treated rats were double-immunostained for BrdU and α-smooth muscle actin (FIG. 12(D)). The data confirmed a marked decrease of proliferating mesangial cells on day 8 after disease induction in all three anti-PDGF-DD antibody treated groups with a maximum of 57% reduction of mesangial cell proliferation. The mesangiolysis scores were similar in anti-PDGF-DD mAb 6.4 and control IgG treated rats (FIG. 12(E)).

Injection of anti-PDGF-DD mAb 6.4 into normal rats did not affect the physiologic glomerular cell turnover as compared to normal rats receiving irrelevant IgG.

Inhibition of PDGF-DD In Vivo Reduces Glomerular Monocyte/Macrophage Influx

On day 5, but not day 8, all three dosages of anti-PDGF-DD mAb 6.4 led to a marked reduction of glomerular monocyte/macrophage influx (FIG. 12(G)). Treatment of normal rats with either the specific anti-PDGF-DD antibody or irrelevant IgG had no effect on the glomerular monocyte/macrophage influx.

Inhibition of PDGF-DD In Vivo Reduces Glomerular Matrix Accumulation

Treatment of the rats with either 10+4 mg anti-PDGF-DD mAb 6.4/kg or 20+8 mg anti-PDGF-DD mAb 6.4/kg resulted in a reduction of glomerular fibronectin accumulation compared to the nephritic controls (FIG. 12(H)). In contrast, glomerular accumulation of type I collagen was not affected by anti-PDGF-DD antibody treatment in either of the three nephritic groups compared to the rats treated with control IgG or PBS (FIG. 12(F)).

In normal rats, glomerular matrix expression was not affected by treatment with anti-PDGF-DD antibody or irrelevant IgG (FIG. 12(H)).

### Example 13

### Efficacy of Anti-PDGF-DD Antibodies In Vivo

The efficacy of anti-PDGF-DD mAb 6.4 to bind PDGF-DD in the anti-Thy-1.1 antibody-induced mesangial proliferative glomerulonephritis model in rats was assessed in vivo as follows.

Male Wistar rats with a normal physiological state, 10 weeks old, and weighing approximately 150-200 g (Charles River, Sulzfeld, Germany) were obtained. The rats were first separated into two groups, normal and those that were to be induced with anti-Thy 1.1 mesangial proliferative glomerulonephritis.

Animals were housed in the local animal facilities as follows: Rats were acclimated for seven (7) days and given food and tap water ad libitum. Animals were examined prior to initiation of the study to assure adequate health and suitability. Animals that were found to be diseased or unsuitable were not assigned to the study. During the course of the study, a 12-hour light/12-hour dark cycle was maintained. A nominal temperature range of 20 to 23° C. with a relative humidity between 30% and 70% was also maintained.

Fully human anti-PDGF-DD mAb 6.4 was generated using Xenomouse® technology as described above. Anti-Thy 1.1 mesangial proliferative glomerulonephritis was induced in the male Wistar rats by injection of 1 mg/kg monoclonal anti-Thy 1.1 antibody. Following the induction of anti-Thy 1.1 nephritis, rats were treated on day 3 and day 5 after disease induction with 10 and 4 mg/kg mAb 6.4 (n=15) or irrelevant human monoclonal antibody (n=15) or PBS (n=15) by daily intraperitoneal injection. The remaining rats were untreated. A total of five groups of rats were studied. After treatment, the rats were analyzed by kidney biopsy, urine albumin, and tissue collection after sacrifice.

1) Fifteen (15) nephritic rats that received a total of 14 mg/kg (10 mg/kg on day 3 and 4 mg/kg on day 5 after disease induction) of anti-PDGF-DD mAb 6.4;

2) Fifteen (15) nephritic rats that received a total of 14 mg/kg (10 mg/kg on day 3 and 4 mg/kg on day 5 after disease induction) of an irrelevant isotype-matched control antibody (not an anti-PDGF-antibody);

3) Fifteen (15) nephritic rats that received 800 μL bolus injections of Tris buffered saline alone;

4) Five (5) normal rats that received a total of 14 mg/kg of anti-PDGF-DD mAb 6.4 (10 mg/kg on day 3 and 4 mg/kg on day 5 after disease induction); and

5) Five (5) normal untreated rats.

Table 5 provides a list of the study design for the five groups that were tested.

TABLE 5
Study Design

| | | | Number of | | | |
|---|---|---|---|---|---|---|
| | | | Animals | | Dose | Volume |
| Group | Type | Treatment | Females | Males | (mg/kg) | (μL) |
| 1 | Nephritic | anti- | 0 | 15 | 10 and 4 | 800 |
| | | PDGF- | | | | |
| | | DD mAb | | | | |
| | | 6.4 | | | | |
| 2 | Nephritic | Irrelevant | 0 | 15 | 10 and 4 | 800 |
| | | antibody | | | | |
| 3 | Nephritic | PBS | 0 | 15 | — | 800 |
| 4 | Normal | anti- | 0 | 5 | 10 and 4 | 800 |
| | | PDGF- | | | | |
| | | DD mAb | | | | |
| | | 6.4 | | | | |
| 5 | Normal | No | 0 | 5 | — | |
| | | treatment | | | | |

The purity of anti-PDGF-DD mAb 6.4 was greater than or equal to 90%. All vials were stored refrigerated, at 4° C. until ready for use. Reserve samples were retained at −80° C. 10 mg/kg and 4 mg/kg body weight were injected intraperitoneally (i.p.) in Tris buffered saline. Dilutions in Tris buffered saline were such that a dose of 10 mg/kg and 4 mg/kg could be administered i.p. in volume of 800 μL. Doses were administered once daily on days 3 and 5 only.

The treatment duration was chosen to treat rats from about one day after the onset to the peak of mesangial cell proliferation, which for OX-7 induced anti-Thy 1.1 nephritis occurred between days 6 and 9 after disease induction.

The rats were observed daily for significant clinical signs, morbidity and mortality approximately 60 minutes after dosing rats. No body weight measurements were performed after initiation of the study. If the animal died prior to necropsy then necropsy and histology data were not included and tissues were not collected.

If an animal died during the necropsy, it was recorded as found dead and necropsy data was not used. However, tissues were collected into formalin for potential evaluation. Animals that were moribund, were killed and treated similarly.

All animals surviving to Day 8 were terminated using cervical dislocation with assessment of gross observations and collection of all scheduled tissues into 10% neutral buffered formalin, Methacarn solution and liquid nitrogen for histomorphologic evaluation.

Staining procedures and tissue preparation: Tissue for light microscopy and immunoperoxidase staining was fixed in methyl Carnoy's solution and embedded in paraffin. Four μm sections were stained with the periodic acid Schiff (PAS) reagent and counterstained with hematoxylin. In the PAS stained sections, the number of mitoses in over 30 cross sections (range 30-100) of consecutive cortical glomeruli containing more than 20 discrete capillary segments each was evaluated by an unbiased observer. Mesangiolysis was graded on a semiquantitative scale as described in Burg et al., Lab Invest 76:505-516 (1997): 0=no mesangiolysis, 1=segmental mesangiolysis, 2=global mesangiolysis, 3=microaneurysm.

Immunoperoxidase Staining: Four μm sections of methyl Carnoy's fixed biopsy tissue were processed by an indirect immunoperoxidase technique as described (Johnson et al, 1990). PDGF-DD was detected by a polyclonal rabbit antibody to human PDGF-D. Primary antibodies were identical to those described previously (Burg et al, 1997; Yoshimura et al, 1991) and included a murine monoclonal antibody (clone 1A4) to α-smooth muscle actin; a murine monoclonal antibody (clone PGF-007) to PDGF-B-chain; a murine monoclonal IgG antibody (clone ED1) to a cytoplasmic antigen present in monocytes, macrophages and dendritic cells; affinity purified polyclonal goat anti-human/bovine type IV collagen IgG preabsorbed with rat erythrocytes; a polyclonal goat antibody to human type I collagen (Southern Biotech Associates, Birmingham, Ala., USA); an affinity purified IgG fraction of a polyclonal rabbit anti-rat fibronectin antibody (Chemicon, Temecula, Calif., USA); plus appropriate negative controls as described previously (Burg et al, 1997; Yoshimura et al, 1991). PDGF-DD was detected by polyclonal rabbit antibody to human PDGF-D. Sera was purified by Protein A Sepharose chromatography. PDGF-C cross reactivity was eliminated by absorption to a PDGF-C affinity column. The resulting immunoglobulin flow through was concentrated and did not react with PDGF-A, B or C by ELISA or western blot analysis. Evaluation of all slides was performed by an observer, who was unaware of the origin of the slides.

To obtain mean numbers of infiltrating leukocytes in glomeruli, more than 50 consecutive cross sections of glomeruli were evaluated and mean values per kidney were calculated. For the evaluation of the immunoperoxidase stains for type I collagen, fibronectin and α-smooth muscle actin each glomerular area was graded semiquantitatively, and the mean score per biopsy was calculated. Each score reflects mainly changes in the extent rather than intensity of staining and depends on the percentage of the glomerular tuft area showing focally enhanced positive staining:

I=0-25%

II=25-50%

III=50-75%

IV=>75%

Immunohistochemical Double-Staining: Double immunostaining for the identification of the type of proliferating cells was performed as reported previously (Kliem et al, 1996; Hugo et al, 1996) by first staining the sections for proliferating cells with a murine monoclonal antibody (clone BU-1) against bromo-deoxyuridine containing nuclease in Tris buffered saline (Amersham, Braunschweig, Germany) using an indirect immunoperoxidase procedure. Sections were incubated with the IgG₁ mAb 1A4 against α-smooth muscle actin and ED1 against monocytes/macrophages. Cells were identified as proliferating mesangial cells or monocytes/macrophages if they showed positive nuclear staining for BrdU and if the nucleus was completely surrounded by cytoplasm positive for α-smooth muscle actin or ED1 antigen. Negative controls included omission of either of the primary antibodies in which case no double-staining was noted.

Urine Measurements: Urinary protein (albuminuria) was measured using the Bio-Rad Protein Assay (Bio-Rad Laboratories GmbH, München, Germany) and bovine serum albumin (Sigma) as a standard. Blood pressure was measured by tail phlethysmography.

Statistical Analysis from numerical data generated, arithmetic means and standard deviations was calculated. Statistical analyses were conducted on data from animals surviving to scheduled termination All values were expressed as means ±SD. Statistical significance (defined as p<0.05) was evaluated using Student t-tests or ANOVA and Bonferroni t-tests. Supplemental analyses were also performed to aid in interpretation of the data, at the discretion of the study director.

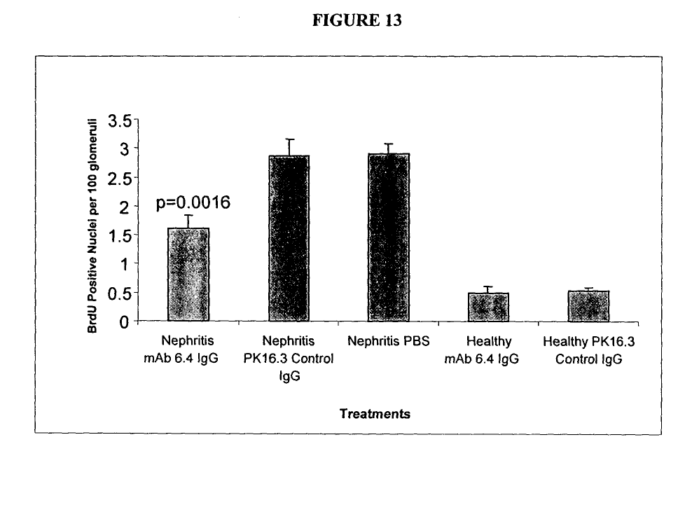

Results: FIG. 13 shows the results of glomerular proliferation as measured by BrdU incorporation in a rat model of glomerulonephritis. Rats were treated with BrdU six hours before sacrifice. BrdU staining of nuclei was measured with anti-BrdU antibody. The number of mitoses observed in a rat model of nephritis were counted per 100 glomeruli. Table 6 summarizes the amount of BrdU Positive Nuclei per 100 glomeruli based on the five groups tested. Table 6, along with the corresponding graph in FIG. 13, demonstrates that administering anti-PDGF-DD mAb 6.4 to animals with nephritis led to less glomerular cells incorporating the thymidine analog BrdU as compared to when irrelevant IgG and PBS were administered. Lane 1 shows the mitotic index of diseased glomeruli. Lane 2 is the rat model of nephritis treated with control antibodies. Lane 3 is the rat model of nephritis treated with PBS. Lane 4 is a healthy rat control treated with anti-PDGF-DD mAb 6.4. Lane 5 is a healthy rat control treated with control antibodies.

TABLE 6
Incorporation of BrdU by glomeruli

| | | | | Mean BrdU | | |
|---|---|---|---|---|---|---|
| | | | | Positive | | |
| | | | | Nuclei per | | p vs. |
| Group | n | Treatment | Day | 100 glomeruli | SD | N + PBS |
| 1: Nephritic | 11 | Anti-PDGF- | 8 | 1.6 | 0.9 | 0.000 |
| | | DD mAb | | | | |
| 2: Nephritic | 14 | Irrelevant | 8 | 2.9 | 1.1 | 1.000 |
| | | Ab | | | | |
| 3: Nephritic | 14 | PBS | 8 | 2.9 | 0.7 | |
| 4: Normal | 5 | Anti-PDGF- | 8 | 0.49 | 0.3 | 0.000 |
| | | DD mAb | | | | |
| 5: Normal | 4 | Irrelevant | 8 | 0.54 | 0.1 | 0.000 |
| | | Ab | | | | |

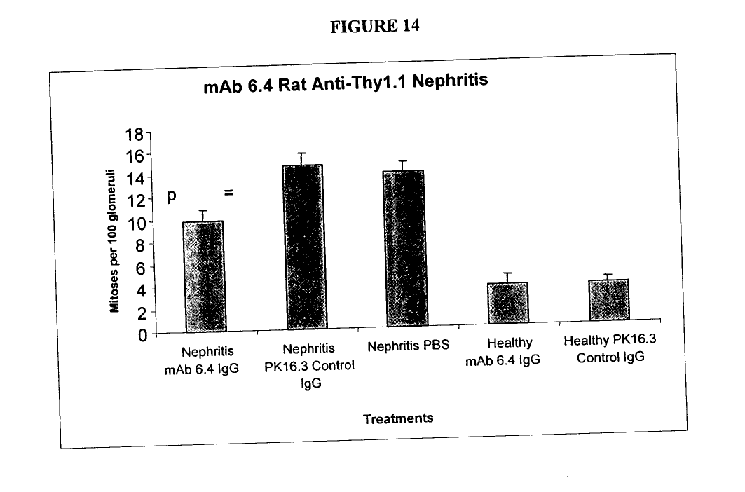

Similarly, FIG. 14 shows the results of glomerular proliferation as measured by PAS stain and quantitation of mitosis in a rat model of glomerulonephritis when treated with anti-PDGF-DD mAb 6.4. Four μM sections were stained with periodic acid-Schiff reagent and counter stained with hemoxylin. Mitoses were measured per 100 glomerular cells. The number of mitoses within 30-50 glomerular tufts was determined. Table 7 shows administration of anti-PDGF-DD mAb 6.4 led to significant reduction of mitoses per 100 glomeruli as compared to irrelevant IgG antibody and PBS. The corresponding graph in FIG. 14 demonstrates that administering anti-PDGF-DD mAb 6.4 to animals with nephritis leads to fewer glomerular cells undergoing mitosis as compared to administering irrelevant IgG and PBS to animals with nephritis. Lane 1 shows the results observed in a rat model of nephritis treated with anti-PDGF-DD mAb 6.4. Lane 2 shows the rat model of nephritis treated with control antibodies. Lane 3 shows the rat model of nephritis treated with PBS. Lane 4 shows a healthy rat control treated with anti-PDGF-DD mAb 6.4. Lane 5 shows a healthy rat control treated with control antibodies.

TABLE 7
Measuring Mitoses (using PAS) in Glomeruli

| | | | | Mean | | |
|---|---|---|---|---|---|---|
| | | | | mitoses per | | |
| | | | | 100 | | p vs. N + |
| Group | n | Treatment | Day | glomeruli | SD | PBS |
| 1: Nephritic | 15 | Anti-PDGF- | 8 | 9.9 | 3.5 | 0.004 |
| | | DD mAb | | | | |
| 2: Nephritic | 15 | Irrelevant | 8 | 14.7 | 3.9 | 0.559 |
| | | Ab | | | | |
| 3: Nephritic | 15 | PBS | 8 | 13.9 | 3.5 | |
| 4: Normal | 5 | Anti-PDGF- | 8 | 3.6 | 2.1 | 0.000 |
| | | DD mAb | | | | |
| 5: Normal | 5 | Irrelevant | 8 | 3.6 | 1.1 | 0.000 |
| | | Ab | | | | |
| 6: Nephritic | 5 | Anti-PDGF- | 5 | 14.7 | 5.6 | 0.373 |
| | | DD mAb | | | | |
| 7: Nephtritic | 5 | Irrelevant | 5 | 22.9 | 15.3 | 0.711 |
| | | Ab | | | | |
| 8: Nephritic | 4 | PBS | 5 | 19.5 | 9.5 | |

As shown in Tables 6 and 7, treatment of nephritic rats with anti-PDGF-DD mAb 6.4 reduced glomerular proliferation as measured by manual count of mitosis (Table 7) as well as by measuring BrdU incorporation (Table 6).

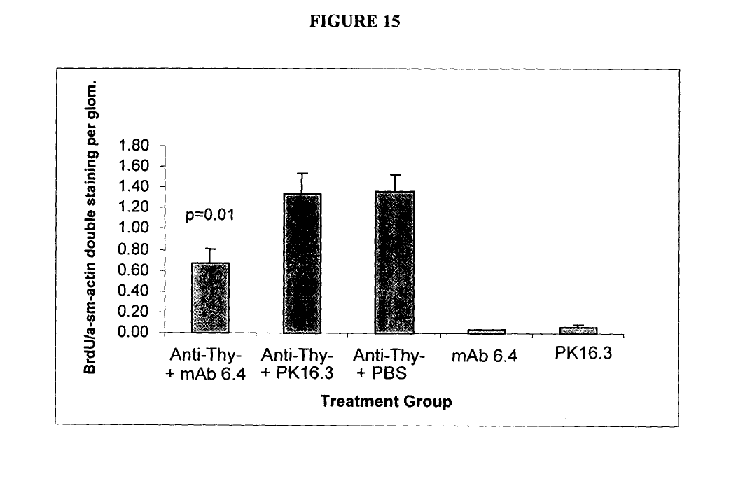

The mesangial cells were also counterstained with anti-smooth muscle actin. The number of mitoses within a given set of mesangial cells (30-50 glomerular tuft) was determined. The results of the double staining assays are provided below in Table 8. Table 8, along with corresponding FIG. 15 demonstrates that anti-PDGF-DD mAb 6.4 is effective at reducing glomerular mitosis as compared to an irrelevant (non-PDGF-D) antibody (PK16.3 IgG) and PBS.

TABLE 8
BrdU + anti-sm-Actin

| | | | | Mean BrdU + | | |
|---|---|---|---|---|---|---|
| | | | | cells per | | p vs. N + |
| Group | n | Treatment | Day | glomeruli | SD | PBS |
| 1: Nephritic | 11 | anti- | 8 | 0.67 | 0.42 | p < 0.05 |
| | | PDGF-D | | | | |
| 2: Nephritic | 14 | IgG | | 1.34 | 0.73 | p < 0.05 |
| 3: Nephritic | 14 | PBS | | 1.37 | 0.56 | |
| 4: Normal | 5 | anti- | | 0.04 | 0.01 | |
| | | PDGF-D | | | | |
| 5: Normal | 4 | IgG | | 0.07 | 0.04 | |

### Example 14

### Dose Responsive Effect of mAb 6.4 on an Acute Rat Thy-1 Model

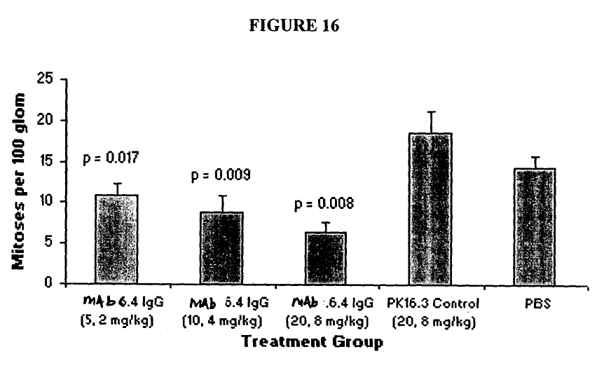

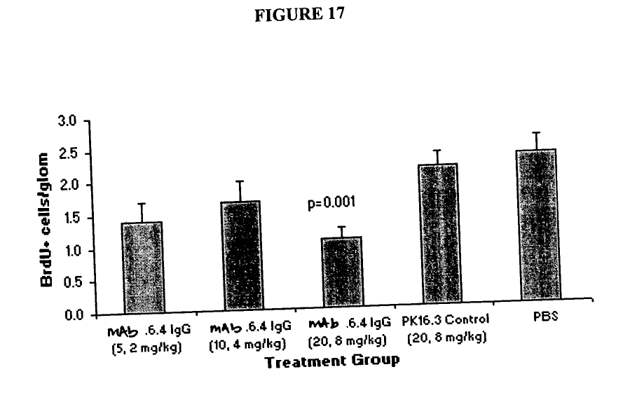

The effect of anti-PDGF-DD mAb 6.4 on Thy-1-induced nephritis was also investigated in a dose responsive manner. Following the induction of anti-Thy 1.1 nephritis, rats were treated from day 3 to 8 after disease induction with 5, 10 and 20 mg/kg followed by 2, 4 and 8 mg/kg respectively of either human anti-PDGF-DD mAb 6.4 (n=15) or irrelevant human PK16.3 monoclonal antibody (n=15) or PBS (n=15) by daily intraperitoneal injection. On day 8 after disease induction antagonism of treatment with anti-PDGF-DD mAb 6.4 led to a significant reduction of mitotic figures per 100 glomeruli, as indicated by FIG. 16 and summarized in Table 9. Treatment with anti-PDGF-DD mAb 6.4 also led to a significant reduction in glomerular cells incorporating the thymidine analog BrdU as indicated by FIG. 17 and summarized in Table 10. Therefore, treatment of a rat anti-Thy-1 model with 10 mg/kg anti-PDGF-DD mAb 6.4 was able to inhibit proliferation by 40 to 70% in a somewhat dose-responsive manner.

TABLE 9

| | | | Mitoses per |
|---|---|---|---|
| Group | Treatment | Dose | 100 glomeruli |
| 1: Nephritic | Anti-PDGF-DD mAb 6.4 | 5 and 2 mg/kg | 10.9 |
| 2: Nephritic | Anti-PDGF-DD mAb 6.4 | 10 and 4 mg/kg | 8.9 |
| 3: Nephritic | Anti-PDGF-DD mAb 6.4 | 20 and 8 mg/kg | 6.5 |
| 4: Nephritic | irrelevant PK16.3 mAb | 20 and 8 mg/kg | 18.6 |
| 5: Nephritic | PBS | — | 14.3 |

TABLE 10

| | | | BrdU + cells |
|---|---|---|---|
| | | | per |
| Group | Treatment | Dose | glomeruli |
| 1: Nephritic | Anti-PDGF-DD mAb 6.4 | 5 and 2 mg/kg | 1.4 |
| 2: Nephritic | Anti-PDGF-DD mAb 6.4 | 10 and 4 mg/kg | 1.6 |
| 3: Nephritic | Anti-PDGF-DD mAb 6.4 | 20 and 8 mg/kg | 1.0 |
| 4: Nephritic | irrelevant PK16.3 mAb | 20 and 8 mg/kg | 2.1 |
| 5: Nephritic | PBS | — | 2.3 |

TABLE 11
Glomerular PDGF-B- and PDGF-D-mRNA expression in
anti-PDGF-D-mAb treated rats

| | PDGF-B mRNA | PDGF-D mRNA |
|---|---|---|
| | [relative to expression in | [relative to expression in |
| Groups | normal rats + control IgG] | normal rats + control IgG] |
| Nephritic + mAb 6.4 | 1.60 (1.3-1.9) | 1.90 (1.5-2.2) |
| 5 mg/kg (day 3) + 2 mg/kg | (n = 2) | (n = 2) |
| (day 5) | | |
| Nephritic + mAb 6.4 | 1.25 (1.0-1.6) | 1.40 (1.2-1.5) |
| 10 mg/kg (day 3) + 4 mg/kg | (n = 3) | (n = 3) |
| (day 5) | | |
| Nephritic + mAb 6.4 | 1.35 (1.1-1.5) | 1.45 (1.2-1.7) |
| 20 mg/kg (day 3) + 8 mg/kg | (n = 2) | (n = 2) |
| (day 5) | | |
| Nephritic + Control IgG | 1.40 (1.1-1.7) | 1.60 (1.4-1.7) |
| 20 mg/kg (day 3) + 8 mg/kg | (n = 3) | (n = 3) |
| (day 5) | | |
| Nephritic + PBS | 1.45 (1.1-1.8) | 2.10 (1.5-2.6) |
| (day 3 and day 5) | (n = 3) | (n = 3) |
| Normal + mAb 6.4 | 0.95 (0.7-1.1) | 1.10 (1.0-1.2) |
| 10 mg/kg (day 3) + 4 mg/kg | (n = 2) | (n = 2) |
| (day 5) | | |
| Normal + Control IgG | 1.0 | 1.0 |
| 10 mg/kg (day 3) + 4 mg/kg | (n = 2) | (n = 2) |
| (day 5) | | |

** Data are means (and ranges) of pooled fractions within each treatment group

### Example 15

### Immunohistochemical Analysis of Human Kidney Disease Tissues

Human kidney disease tissues were tested for the presence of PDGF-DD by immunohistochemical analysis.

Immunohistochemical staining was performed with rabbit anti-PDGF-DD IgG that does not recognize PDGF-AA, PDGF-BB or PDGF-CC. Staining was followed by detection with goat anti-rabbit conjugated to horseradish peroxidase (anti-rabbit-HRP conjugate). After incubation with anti-rabbit-HRP conjugate, a solution of diaminobenzidine (DAB) was applied onto the sections to visualize the immunoreactivity.

In active glomerular nephritis, tubule (most likely proximal) staining was evident. Some glomeruli also stained positive (about 10-20% of mesangiums/field). A tissue from a patient with chronic allograft rejection also stained positive showing tubule and vascular staining. Cellular deposits were also detected in the mesangium suggestive of proinflammatory mast cells and cellular deposition. Control rabbit IgG did not stain.

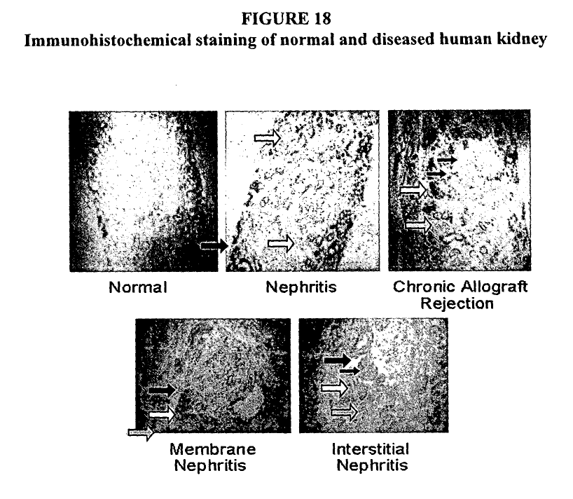

PDGF-DD staining was also evident in drug-induced interstitial nephritis. Here, increased tubular staining, prominent staining of mesangial cells, and some staining of infiltrating proinflammatory cells was observed. No significant staining of normal human kidney was observed except perhaps very weak tubular staining. In nephrosclerosis, PDGF-DD staining of tubules was noted (data not shown). No staining was observed in ischemic tubular injury (data not shown). These results suggest elevation of PDGF-DD in many human kidney pathologies, suggestive of its role in kidney disease. PDGF-DD may be involved in changes in tubular interstitium, mesangial proliferation, and active inflammatory processes (see FIG. 18). White and gray arrows depict capillary and tubule staining respectively. Small black arrows show punctate inflammatory cell deposits in mesangium.

### Example 16

### Analyzing the Risk for Developing, the Diagnosis of, and Staging of Nephritis with ELISA

Serum levels of PDGF-DD from patients afflicted with nephritis is analyzed. The concentration of PDGF-DD is assessed using a quantitative sandwich ELISA with 2 fully human mAbs raised against PDGF-DD. It is found that PDGF-DD levels are elevated four to seven fold in the sera of nephritis patients compared to normal patients. These differences in the level of PDGF-DD can accordingly help form diagnostics and help practitioners track staging of nephritis and related diseases.

### Example 17

### Treatment of Nephritis in a Human with Anti-PDGF-DD Antibodies

A practitioner administers an effective amount of anti-PDGF-DD antibodies to a patient in need, such that the patient in need has symptomatic relief or the nephritis is effectively treated. The administration and dosage is specific to the patient. The administration of the anti-PDGF-DD antibodies is through subcutaneous injection.

The various methods and techniques described above provide a number of ways to carry out numerous embodiments. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the methods described herein have been disclosed in the context of certain embodiments and examples, it will be understood by those skilled in the art that these methods extend beyond the specifically disclosed embodiments to other alternative embodiments and/or uses and obvious modifications and equivalents thereof. Accordingly, the methods described herein are not intended to be limited by the specific disclosures of preferred embodiments herein, but instead by reference to claims attached hereto.

What is claimed is:

1. A method of effectively treating nephritis, comprising:
selecting an animal in need of treatment for nephritis; and
administering to said animal a therapeutically effective dose of an antibody, or binding fragment thereof, that binds to platelet derived growth factor-DD (PDGF-DD).

2. The method of claim 1, wherein said animal is a human.

3. The method of claim 1, wherein said antibody is a fully human monoclonal antibody.

4. The method of claim 1, wherein said nephritis is selected from the group consisting of: mesangial proliferative nephritis, mesangial proliferative glomerulonephritis, mesangiocapillary glomerulonephritis, systemic lupus erythematosus, glomerular nephritis, progressive renal disease, renal interstital fibrosis, renal failure, and diabetic nephropathy.

5. The method of claim 1, wherein the nephritis is related to proliferation of glomerular or mesangial cells.

6. The method of claim 1, wherein said administration is via subcutaneous injection.

7. The method of claim 1, wherein said administration is via intramuscular injection.

8. A method of inhibiting mesangial cell proliferation, comprising:
providing a monoclonal antibody, or binding fragment thereof, that binds platelet derived growth factor-DD (PDGF-DD); and
contacting proliferating mesangial cells with said monoclonal antibody under conditions that result in inhibited proliferation of said cells.

9. The method of claim 8, wherein said antibody is a fully human monoclonal antibody.

10. The method of claim 8, wherein said mesangial cells are human mesangial cells.

11. A method of effectively treating mesangial proliferative glomerulonephritis, comprising:
selecting an animal in need of treatment for mesangial proliferative glomerulonephritis; and
administering to said animal a therapeutically effective dose of an antibody, or binding fragment thereof, that binds to platelet derived growth factor-DD (PDGF-DD).

12. The method of claim 11, wherein said animal is a human.

13. The method of claim 11, wherein said antibody is a fully human monoclonal antibody.

14. The method of claim 11, wherein said administration is via subcutaneous injection.

15. The method of claim 11, wherein said administration is via intramuscular injection.

16. A method of detecting nephritis, comprising:
selecting a patient at risk for nephritis;
contacting a renal cell from said patient with an antibody, or binding fragment thereof, that binds PDGF-DD; and
detecting binding of said cells and said antibody, wherein a detectable binding is indicative of nephritis.

17. The method of claim 16, wherein said antibody is a monoclonal antibody.

18. The method of claim 16, wherein said antibody is a fully human monoclonal antibody.

19. The method of claim 16, wherein said antibody is labelled with a marker selected from the group consisting of: a fluorochrome, an enzyme, a radionuclide and a radiopaque material.

20. The method of claim 16, wherein said binding fragment comprises a Fab′ fragment.

21. The method of claim 16, wherein said nephritis is selected from the group consisting of: mesangial proliferative nephritis, mesangial proliferative glomerulonephritis, mesangiocapillary glomerulonephritis, systemic lupus erythematosus, glomerular nephritis, progressive renal disease, renal interstital fibrosis, renal failure, and diabetic nephropathy.
